(19) United States
(12) Patent Application Publication
(10) Pub. No.: US 2023/0414659 A1
(43) Pub. Date: Dec. 28, 2023

(54) METHODS OF ADMINISTERING GENETICALLY MODIFIED B CELLS FOR IN VIVO DELIVERY OF THERAPEUTIC AGENTS

(71) Applicant: Immusoft Corporation, Seattle, WA (US)

(72) Inventor: Christiane S. HAMPE, Seattle, WA (US)

(21) Appl. No.: 18/034,865

(22) PCT Filed: Oct. 29, 2021
(86) PCT No.: PCT/US2021/057363

§ 371 Date: May 1, 2023

Related U.S. Application Data
(60) Provisional application No. 63/107,992, filed on Oct. 30, 2020.

(51) Int. Cl. A61K 35/17; C12N 5/0781; C12N 9/24; A61P 3/00; A61K 9/00

(52) U.S. Cl. CPC A61K 35/17; C12N 5/0635; C12N 9/2402; C12Y 302/01075; A61P 3/00; A61K 9/0085; C12N 2510/00

(57) ABSTRACT

Provided herein are methods for administering engineered B cells to produce a therapeutic agent in vivo. In various embodiments, engineered B cells are directly administered to the central nervous system (CNS). The compositions and methods disclosed herein may be used for enzyme replacement therapy, for example, treatment of diseases or disorders associated with lysosomal storage dysfunction through production of iduronidase (IDUA).

### RELATED APPLICATIONS

This application is a U.S. national phase application of International PCT Application No. PCT/US2021/057363, filed Oct. 29, 2021, which claims the benefit under 35 U.S.C. § 119(e) to U.S. Provisional Application No. 63/107,992, filed Oct. 30, 2020, the contents of which are incorporated herein by reference in their entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is IMCO-009_01US_ST25.txt. The text file is about 10,137 bytes, was created on Apr. 28, 2023, and is being submitted electronically via EFS-Web.

### TECHNICAL FIELD

This disclosure relates generally to methods for administering engineered cells to a subject to produce a therapeutic agent, such as a therapeutic protein. Specifically disclosed are methods for administering engineered B cells to the central nervous system.

### BACKGROUND

Cellular therapy in the CNS (e.g., brain) presents unique challenges as the CNS is immunologically privileged. For this reason, prior efforts at local administration of B cells have not generally extended to administration to the CNS (e.g., brain).

### BRIEF SUMMARY

The present disclosure provides methods for administering genetically modified (engineered) B cells directly to the central nervous system (CNS) of a subject for treating chronic diseases and disorders affecting such tissues.

In an aspect, the disclosure provides a method of administering genetically modified B cells to a subject for in vivo production of a therapeutic agent comprising: administering one or more doses of genetically modified B cells to a subject's central nervous system (CNS).

In some embodiments, the administering comprises infusion into the cerebrospinal fluid (CSF) of the subject.

In some embodiments, the administering comprises intracisternal injection.

In some embodiments, the administering comprises intrathecal injection.

In some embodiments, the administering comprises intracerebroventricular injection (ICV).

In some embodiments, the intracerebroventricular injection (ICV) occurs in one or more brain cavities.

In some embodiments, the one or more brain cavities is a lateral ventricle.

In some embodiments, the one or more brain cavities is a third ventricle.

In some embodiments, the one or more brain cavities is a cerebral aqueduct.

In some embodiments, the one or more brain cavities is a fourth ventricle.

In some embodiments, the therapeutic agent produced by the genetically modified B cells is iduronidase (IDUA).

In some embodiments, doses comprise the genetically modified B cells at sub-optimal single-dose concentrations, wherein sub-optimal single dose concentrations are determined by: (i) testing multiple single doses of the modified B cells; (ii) determining an optimal single-dose concentration of the modified B cells, wherein increasing the dosage of modified B cells present in a single-dose concentration of modified B cells results in the production of the therapeutic agent; (iii) testing multiple sub-optimal single dose concentrations of the modified B cells; and (iv) determining a sub-optimal single-dose of the modified B cells, wherein the resulting dosage results in a greater than linear increase over lower dosages, wherein the sub-optimal single-dose concentration is less than or equivalent to about one half or about one third the dose of the optimal single-dose concentration.

In some embodiments, the administering optionally comprises one or more sequential doses of the genetically modified B cells.

In some embodiments, the subject is a mammal.

In some embodiments, the subject is a human

In some embodiments, the genetically modified B cells are autologous to the subject.

In some embodiments, the genetically modified B cells are allogeneic to the subject.

In some embodiments, the therapeutic agent is a protein.

In some embodiments, the protein is an enzyme.

In some embodiments, the genetically modified B cells are CD20−, CD38+, and CD138+.

In some embodiments, the genetically modified B cells are CD20−, CD38+, and CD138−.

In some embodiments, the genetically modified B cells were prepared using a Sleeping Beauty transposon to express the therapeutic agent in the B cells.

In some embodiments, the genetically modified B cells were prepared using a recombinant viral vector to express the therapeutic agent in the B cells.

In some embodiments, the recombinant viral vector encodes a recombinant retrovirus, recombinant lentivirus, recombinant adenovirus, or recombinant adeno-associated virus. In some embodiments, the genetically modified B cells were prepared by gene editing of the B cell genome or by targeted integration into the genome of the B cell of a polynucleotide sequence encoding the therapeutic agent.

In some embodiments, the targeted integration comprises a zinc finger nuclease-mediated gene integration, CRISPR-mediated gene integration or gene editing, TALE-nuclease-mediated gene integration, or meganuclease-mediated gene integration.

In some embodiments, the targeted integration of polynucleotide occurred via homologous recombination.

In some embodiments, the targeted integration comprises a viral vector-mediated delivery of a nuclease capable of inducing a DNA cleavage at a target site.

In some embodiments, the nuclease is a zinc finger nuclease, a Cas nuclease, a TALE-nuclease, or a meganuclease.

In some embodiments, the genetically modified B cell comprise a polynucleotide having a sequence that is identical to SEQ ID NO: 1.

In some embodiments, the genetically modified B cells comprise a polynucleotide having a sequence that is at least about 85% identical to SEQ ID NO: 1, or at least about 90%, 95%, 96%, 97%, 98%, 99%, or greater than 99% identical to SEQ ID NO: 1.

In some embodiments, the genetically modified B cells are engineered on Day 2 or Day 3 after the start of culturing.

In some embodiments, the genetically modified B cells are engineered using a method comprising electroporation.

In some embodiments, the genetically modified B cells are harvested for administration to a subject on Day 4, Day 5, Day 6, or Day 7 in culture after engineering.

In some embodiments, the genetically modified B cells are harvested for administration to a subject on Day 8 or later in culture after engineering.

In some embodiments, the genetically modified B cells are harvested for administration to a subject on Day 10 or earlier in culture after engineering.

In some embodiments, the harvested genetically modified B cells do not produce significant levels of inflammatory cytokines.

In some embodiments, the genetically modified B cells are harvested at a time-point in culture at which it is determined that they do not produce significant levels of inflammatory cytokines.

In some embodiments, the genetically modified B cells are grown in a culture system that comprises each of IL-2, IL-4, IL-10, IL-15, IL-21, and a multimerized CD40 ligand throughout the entire culture period pre- and post-engineering.

In some embodiments, the multimerized CD40 ligand is a HIS tagged CD40 ligand that is multimerized using an anti-his antibody.

In some embodiments, the method comprises expanding the genetically modified B cells prior to the administering to the subject.

In some embodiments, the final population of expanded genetically modified B cells demonstrates a high degree of polyclonality.

In some embodiments, any particular B cell clone in the final population of expanded genetically modified B cells comprises less than 0.2% of the total B cell population.

In some embodiments, any particular B cell clone in the final population of expanded genetically modified B cells comprises less than 0.05% of the total B cell population.

In some embodiments, the genetically modified B cells comprise a polynucleotide encoding a human DHFR gene with enhanced resistance to methotrexate.

In some embodiments, the human DHFR gene with enhanced resistance to methotrexate contains a substitution of leucine to tyrosine at amino acid 22 and a substitution of phenylalanine to serine at amino acid 31.

In some embodiments, the method comprises treating the genetically modified B cells with methotrexate prior to harvesting for administration.

In some embodiments, the methotrexate treatment is between 100 nM and 300 nM.

In some embodiments, the methotrexate treatment is 200 nM.

In some embodiments, the genetically modified B cells travel throughout tissues within the central nervous system (CNS) upon administration to the subject.

In some embodiments, the administration of the genetically modified B cells to the subject results in the reduction of glycosaminoglycans (GAGs) in diverse tissues of the subject.

In some embodiments, the administration of the genetically modified B cells to the subject results in the reduction of GAGs in tissues within the central nervous system (CNS).

In some embodiments, the genetically modified B cells persist in the central nervous system (CNS) for at least about one week, two weeks, three weeks, four weeks, five weeks, or six weeks.

In some embodiments, the genetically modified B cells persist in the central nervous system (CNS) for at least about one month, two months, three months, four months, five months, or six months.

In some embodiments, the genetically modified B cells persist in the central nervous system (CNS) for at most about one week, two weeks, three weeks, four weeks, five weeks, or six weeks.

In some embodiments, the genetically modified B cells persist in the central nervous system (CNS) for at most about one month, two months, three months, four months, five months, or six months.

In some embodiments, the subject has a disease or disorder associated with lysosomal storage dysfunction.

In some embodiments, the disease or disorder associated with lysosomal storage dysfunction is caused by enzyme alpha-L-iduronidase (IDUA) deficiency.

In some embodiments, the subject has mucopolysaccharidosis type I (MPS I).

In some embodiments, the administration of the genetically modified B cells treats the subject's disease or disorder associated with lysosomal storage dysfunction.

In some embodiments, the administration of the genetically modified B cells treats the subject's MPS I.

Other aspects and advantages of the invention will be readily apparent from the following detailed description of the invention.

### BRIEF DESCRIPTION OF FIGURES

For the purpose of illustrating the disclosure, there are depicted in the drawings of certain embodiments of the disclosure. However, the disclosure is not limited to the precise arrangements and instrumentalities of the embodiments depicted in the drawings.

FIG. 1 depicts analysis of IDUA activity from whole brain tissue of mice administered engineered B cells directly to the central nervous system.

FIG. 2 depicts analysis of GAG from brain tissue of mice administered engineered B cells directly to the central nervous system.

FIG. 3 shows bioluminescence imaging of NSG mice ICV injected with LUC-transposed human B cells. Mice were imaged biweekly (IVIS). Luminescence intensity is indicated on the scale.

FIG. 4 shows a line graph of the bioluminescence imaging results from FIG. 3.

### DETAILED DESCRIPTION

Enzyme deficiency can result in chronic diseases and disorders. Enzyme replacement therapy is a current method for treating such diseases and disorders by direct infusion of an exogeneous enzyme (therapeutic agent) to counteract the enzyme deficiency. However, this method of treating has disadvantages. The efficacy of injected recombinant therapeutic protein is limited by the finite half-life of the protein, and can provide sub-optimal tissue penetration by the therapeutic agent. The present disclosure addresses some of the limitations of enzyme replacement therapy to more effectively treat particular diseases and disorders associated with enzyme deficiency.

The use of differentiated B cell compositions for long term in vivo expression of a transgene has been identified as a promising strategy for the treatment of various diseases and disorders, including enzyme deficiency. However, methods for administering modified B cells for delivery of therapeutic agents have not yet been described in order to achieve therapeutically effective levels of the agents in vivo. The present disclosure provides methods of administering a genetically modified (engineered) B cell comprising expression of a transgene encoding a modified human DHFR for production of iduronidase (IDUA) in vivo for treating chronic diseases and disorders, particularly diseases and disorders associated with lysosomal storage dysfunction, by directly administering the engineered B cells to the central nervous system (CNS).

The embodiments described herein relate, in part, to the inventors' surprising discovery that administration of differentiated B cell compositions by direct injection into the CNS results in prolonged B cell survival and expression of the transgene in the CNS. Moreover, the expressed transgene resulted in a pharmacologic effect in the brain.

### Definitions

To facilitate understanding of the invention, a number of terms and abbreviations as used herein are defined below as follows:

As used herein the singular forms “a”, “an” and “the” include plural aspects unless the context clearly dictates otherwise. For example, reference to “a vector” includes a single vector, as well as two or more vectors; reference to “a cell” includes one cell, as well as two or more cells; and so forth.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference to the term “e.g.” is intended to mean “e.g., but not limited to” and thus it should be understood that whatever follows is merely an example of a particular embodiment, but should in no way be construed as being a limiting example. Unless otherwise indicated, use of “e.g.” is intended to explicitly indicate that other embodiments have been contemplated and are encompassed by the present invention.

Reference throughout this specification to “embodiment” or “one embodiment” or “an embodiment” or “some embodiments” or “certain embodiments” means that a particular feature, structure or characteristic described in connection with the embodiment is included in at least one embodiment of the present invention. Thus, the appearances of the phrases “in one embodiment” or “in an embodiment” or “in certain embodiments” or “in some embodiments” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. Similarly, a “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

As used herein, “optional” or “optionally” means that the subsequently described event, or circumstances, may or may not occur, and that the description includes instances where said event or circumstance occurs and instances in which it does not.

As used herein, “substantially” or “essentially” means of ample or considerable amount, quantity, size; nearly totally or completely; for instance, 95% or greater of some given quantity.

The terminology used herein is for the purpose of describing particular embodiments only and is not intended to be limiting. Unless defined otherwise, all technical and scientific terms used herein have the same meanings as commonly understood by one of ordinary skill in the art to which the present disclosure belongs. Any materials and methods similar or equivalent to those described herein can be used to practice the present invention. The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J.B. Lippincott Company, 1993).

The terms “in vitro”, “ex vivo”, and “in vivo” are intended herein to have their normal scientific meanings. Accordingly, e.g., “in vitro” is meant to refer to experiments or reactions that occur with isolated cellular components, such as, e.g., an enzymatic reaction performed in a test tube using an appropriate substrate, enzyme, donor, and optionally buffers/cofactors. “Ex vivo” is meant to refer to experiments or reactions carried out using functional organs or cells that have been removed from or propagated independently of an organism. “in vivo” is meant to refer to experiments or reactions that occur within a living organism in its normal intact state.

As used herein, “mammal” includes humans and both domestic animals such as laboratory animals and household pets, (e.g., cats, dogs, swine, cattle, sheep, goats, horses, and rabbits), and non-domestic animals such as wildlife and the like.

As used herein, “subject,” includes any animal that exhibits a disease or symptom, or is at risk for exhibiting a disease or symptom, which can be treated with an agent of the invention. Suitable subjects include laboratory animals (such as mouse, rat, rabbit, or guinea pig), farm animals, and domestic animals or pets (such as a cat or dog). Non-human primates and, preferably, human patients, are included.

As used herein, “proliferation” or “expansion” refers to the ability of a cell or population of cells to increase in number.

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g. growth factors and morphogenic factors) to induce specified lineage commitment, or differentiation, of said stem cells. For example, a memory B cell that transitions to a plasma cell is differentiated.

As used herein, the term “CD20−, CD38+, and CD138+” is used to refer to cells that express both the CD38 and the CD138 surface markers and do not express CD20 surface marker, wherein “+” denotes presence and “−” denotes absence. Thus, alternatively, the term “are CD20−, CD38+, and CD138−” is used to refer to cells that express CD38 surface marker and do not express both the CD38 and CD138 surface markers.

B cells are specific immune cells that act, as antigen presenting cells (APC) and internalize antigens. Antigens are taken up by the B cell through receptor-mediated endocytosis and processed. Antigens are processed into antigenic peptides, loaded onto MHC II molecules, and presented on the B cell extracellular surface to CD4+ T helper cells. These T cells bind to the MHC II/antigen molecule and cause activation of the B cell. Upon stimulation by a T cell, the activated B cell begins to differentiate into more specialized cells. Germinal center B cells may differentiate into long-lived memory B cells or plasma cells. Further, secondary immune stimulation may result in the memory B cells giving rise to additional plasma cells. The formation of plasma cells from either memory or non-memory B cells is preceded by the formation of precursor plasmablasts that eventually differentiate into plasma cells, which produce large volumes of antibodies (see e.g., Trends Immunol. 2009 June; 30(6): 277-285; Nature Reviews, 2005, 5:231-242). Plasmablasts secrete more antibodies than B cells, but less than plasma cells. They divide rapidly, and they continue to internalize antigens and present antigens to T cells. Plasmablasts have the capacity to migrate to sites of chemokine production (e.g. in bone marrow) whereby they may differentiate into long-lived plasma cells. Ultimately, a plasmablast may either remain as a plasmablast for several days and then die or irrevocably differentiate into a mature, fully differentiated plasma cell. Specifically, plasmablasts that are able home to tissues containing plasma cell survival niches (e.g., in bone marrow) are able to displace resident plasma cells in order to become long lived plasma cells, which may continue to secrete high levels of proteins for years. Terminally differentiated plasma cells typically do not express common pan-B cell markers, such as CD19 and CD20, and express relatively few surface antigens. Plasma cells express CD38, CD78, CD138 and interleukin-6 receptor (IL-6R) and lack expression of CD45, and these markers can be used, e.g., by flow cytometry, to identify plasma cells. CD27 is also a good marker for plasma cells as naive B cells are CD27−, memory B cells are CD27+ and plasma cells are CD27++. Memory B cell subsets may also express surface IgG, IgM and IgD, whereas plasma cells do not express these markers on the cell surface. CD38 and CD138 are expressed at high levels on plasma cells (See Wikipedia, The Free Encyclopedia., “Plasma cell” Page Version ID: 404969441; Date of last revision: 30 Dec. 2010 09:54 UTC, retrieved Jan. 4, 2011; See also: Jourdan et al. Blood. 2009 Dec. 10; 114(25):5173-81; Trends Immunol. 2009 June; 30(6):277-285; Nature Reviews, 2005, 5:231-242; Nature Med. 2010, 16: 123-129; Neuberger, M. S.; Honjo, T.; Alt, Frederick W. (2004). Molecular biology of B cells. Amsterdam: Elsevier, pp. 189-191, Bertil Glader; Greer, John G; John Foerster; Rodgers, George G.; Paraskevas, Frixos (2008). Wintrobe's Clinical Hematology, 2-Vol. Set. Hagerstown, MD; Lippincott & Wilkins. pp. 347; Walport, Mark; Murphy, Kenneth; Janeway, Charles; Travers, Paul J. (2008). Janeway's immunobiology. New York: Garland Science, pp. 387-388; Rawstron A C (May 2006). “Immunophenotyping of plasma cells”. Curr Protoc Cytom).

The B cells used in the methods described herein include pan B cells, memory B cells, plasmablasts, and/or plasma cells. In one embodiment, the modified B cells are memory B cells. In one embodiment, the modified B cells are plasmablasts. In one embodiment, the modified B cells are plasma cells.

As used herein, the term “isolated” is used to refer to molecules or cells that are removed from native environments. As used herein, the term “non-naturally occurring” is used to refer to isolated molecules or cells that possess markedly different structures than counterparts found in nature.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

As used herein, “antibody” is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or VH) and a heavy chain constant region. The heavy chain constant region typically comprises three domains—CH1, CH2 and CH3. Each light chain comprises a light chain variable region (which may be abbreviated as LCVR, VL, VK, VK or VL) and a light chain constant region. The light chain constant region will typically comprise one domain (CL1). The VH and VL regions can be further subdivided into regions of hypervariability, termed complementarity determining regions (CDRs), interspersed with regions that are more conserved, also referred to as framework regions (FR).

The terms “host”, “host cell”, “host cell line” and “host cell culture” are used interchangeably and refer to cells into which exogenous nucleic acid has been introduced, including the progeny of such cells. Host cells include “transformants” or “transformed cells” or “engineered cells”, which include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as, but not limited to, B cells.

As used herein, the terms “vector” and “construct”, which are used interchangeably, may be nucleic acid molecules, preferably DNA molecules derived, for example, from a plasmid, bacteriophage, or virus, into which a nucleic acid sequence may be inserted or cloned. A vector may contain one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art. In some embodiments, vectors are used to generate the engineered NK cell or the engineered macrophage cell of the current invention.

As used herein, an “expression construct” refers to a nucleic acid molecule which comprises coding sequences for the therapeutic protein, promoter, and may include other regulatory sequences therefor, which cassette may be engineered into a genetic element and/or packaged into the capsid of a viral vector (e.g., a viral particle). Typically, such an expression cassette for generating a viral vector contains the construct sequences described herein flanked by packaging signals of the viral genome and other expression control sequences such as those described herein. Any of the expression control sequences can be optimized for a specific species using techniques known in the art including, e.g., codon optimization.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

As used herein, “biological activity” or “bioactivity” refers to any response induced in an in vitro assay or in a cell, tissue, organ, or organism, (e.g., an animal, or a mammal, or a human) as the result of administering any compound, agent, polypeptide, conjugate, pharmaceutical composition contemplated herein. Biological activity may refer to agonistic actions or antagonistic actions. The biological activity may be a beneficial effect; or the biological activity may not be beneficial, i.e. a toxicity. In some embodiments, biological activity will refer to the positive or negative effects that a drug or pharmaceutical composition has on a living subject, e.g., a mammal such as a human. Accordingly, the term “biologically active” is meant to describe any compound possessing biological activity, as herein described. Biological activity may be assessed by any appropriate means currently known to the skilled artisan. Such assays may be qualitative or quantitative. The skilled artisan will readily appreciate the need to employ different assays to assess the activity of different polypeptides; a task that is routine for the average researcher. Such assays are often easily implemented in a laboratory setting with little optimization requirements, and more often than not, commercial kits are available that provide simple, reliable, and reproducible readouts of biological activity for a wide range of polypeptides using various technologies common to most labs. When no such kits are available, ordinarily skilled researchers can easily design and optimize in-house bioactivity assays for target polypeptides without undue experimentation; as this is a routine aspect of the scientific process.

“Therapeutic agent” refers to any compound that, when administered to a subject, (e.g., preferably a mammal, more preferably a human), in a therapeutically effective amount is capable of effecting treatment of a disease or condition as defined below.

As used herein, the term “treat” or “treating” or “treatment” embraces at least an amelioration of the symptoms associated with a disease or condition in the patient, where amelioration is used in a broad sense to refer to at least a reduction in the magnitude of a parameter, e.g. a symptom associated with the condition being treated. As such, “treatment” as used herein covers the treatment of the disease or condition of interest in a subject, preferably a human, having the disease or condition of interest, and includes: (i) preventing or inhibiting the disease or condition from occurring in a subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady, injury or condition may not have a known causative agent (so that etiology has not yet been worked out), and it is, therefore, not yet recognized as an injury or disease but only as an undesirable condition or syndrome, wherein a more or less specific set of symptoms have been identified by clinicians.

As used herein, “therapeutically effective” refers to an amount of engineered B cell or therapeutic agent that is sufficient to treat or ameliorate, or in some manner reduce the symptoms associated with a disease or disorder, such as enzyme deficiency, protein deficiency, hormone deficiency, inflammation, cancer, autoimmunity, or infection. When used with reference to a method, the method is sufficiently effective to treat or ameliorate, or in some manner reduce the symptoms associated with a disease or condition. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

The term “combination” refers to either a fixed combination in one dosage unit form, or a kit of parts for the combined administration where engineered B cells and therapeutic agent and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

### Administering Genetically Modified B Cells

The present disclosure relates generally to B cells that have been altered through introduction of nucleic acids to produce a therapeutic agent and methods of administering the modified B cells. In some embodiments, the terms “engineered B cell”, “genetically engineered B cell”, “modified B cell” and “genetically modified B cell” are used interchangeably herein to refer to such altered B cells that comprises one or more nucleic acids (e.g., a transgene) to produce a therapeutic agent (e.g., a transgene that enables expression of a polypeptide such as a therapeutic polypeptide).

Accordingly, the methods for administering modified B cell described herein are useful for long term in vivo delivery of the B cells to the CNS and expression of therapeutic agents in the CNS. The present disclosure provides methods for achieving sufficient enrichment and number of cells producing a therapeutic agent and achieving sufficient levels of the therapeutic agent in the CNS while ensuring product safety.

As used herein, the phrases “long term in vivo survival” and “long term survival” refer to the survival of the modified B cells described herein for 10 or more days post administration in a subject. Long term survival may be measured in days, weeks, or even years. In some embodiments, a majority of the modified B cells survive in vivo for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more days post-administration. In some embodiments, a majority of the modified B cells survive in vivo for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more weeks post-administration. In some embodiment, the modified B cells survive in vivo for 1, 1.5, 2, 2.5, 3, 3.5, 4, 4.5, 5, 6, 7, 8, 9, 10, 11, 12, 13, 14, 15, 16, 17, 18, 19, 20, 25, 30 or more years. Additionally, while the modified B cells described herein may survive in vivo for 10 or more days, it is understood that a majority of the modified B cells survive in vivo for 1, 2, 3, 4, 5, 6, 7, 8, 9 or more days post-administration. Accordingly, it is contemplated that modified B cells described herein are useful for short-term treatment (e.g., 7 days) and long-term treatment (e.g., 30 or more days) methods. In some embodiments, the genetically modified B cells persist in the central nervous system (CNS) for at least about one week, two weeks, three weeks, four weeks, five weeks, or six weeks. In some embodiments, the genetically modified B cells persist in the central nervous system (CNS) for at least about one month, two months, three months, four months, five months, or six months. In some embodiments, the genetically modified B cells persist in the central nervous system (CNS) for at most about one week, two weeks, three weeks, four weeks, five weeks, or six weeks. In some embodiments, the genetically modified B cells persist in the central nervous system (CNS) for at most about one month, two months, three months, four months, five months, or six months.

In an aspect, the disclosure provides a method of administering genetically modified B cells to a subject for in vivo production of a therapeutic agent comprising: administering one or more doses of genetically modified B cells to a subject's central nervous system (CNS).

In some embodiments, the therapeutic agent produced by the genetically modified B cells is iduronidase (IDUA).

In some embodiments, the administering comprises infusion into the cerebrospinal fluid (CSF) of the subject. In some embodiments, the administering comprises intracisternal injection. In some embodiments, the administering comprises intrathecal injection. In some embodiments, the administering comprises intracerebroventricular injection (ICV). In some embodiments, the intracerebroventricular injection (ICV) occurs in one or more brain cavities. In some embodiments, the one or more brain cavities is a lateral ventricle. In some embodiments, the one or more brain cavities is a third ventricle. In some embodiments, the one or more brain cavities is a cerebral aqueduct. In some embodiments, the one or more brain cavities is a fourth ventricle.

The modified B cells can be administered as a single dosage or multiple dosages. In some embodiments, doses comprise the genetically modified B cells at sub-optimal single-dose concentrations, wherein sub-optimal single dose concentrations are determined by: (i) testing multiple single doses of the modified B cells; (ii) determining an optimal single-dose concentration of the modified B cells, wherein increasing the dosage of modified B cells present in a single-dose concentration of modified B cells results in the production of the therapeutic agent; (iii) testing multiple sub-optimal single dose concentrations of the modified B cells; and (iv) determining a sub-optimal single-dose of the modified B cells, wherein the resulting dosage results in a greater than linear increase over lower dosages, wherein the sub-optimal single-dose concentration is less than or equivalent to about one half or about one third the dose of the optimal single-dose concentration. In some embodiments, the administering optionally comprises one or more sequential doses of the genetically modified B cells.

The optimal dosage and treatment regime for a particular subject can be determined by one skilled in the art of medicine by monitoring the patient for signs of disease and adjusting the treatment accordingly. The treatment may also be adjusted after measuring the levels of a therapeutic agent (e.g., a gene or protein of interest) in a biological sample (e.g., body fluid or tissue sample) can also be used to assess the treatment efficacy, and the treatment may be adjusted accordingly to increase or decrease.

In some aspects of the present disclosure, an optimal dosage of the modified B cells for a multi-dose regime may be determined by first determining an optimal single-dose concentration of the B cells for a subject, decreasing the number of B cells present in the optimal single-dose concentration to provide a sub-optimal single- dose concentration of the modified B cells, and administering two or more dosages of the sub-optimal single-dose concentration of modified B cells to the subject. In some aspects, 2, 3, or more dosages of a sub-optimal single-dose concentration of modified B cells are administered to the subject. In some aspects, the administration of 2, 3, or more dosages of a sub-optimal single-dose concentration of modified B cells to a subject results in synergistic in vivo production of a therapeutic polypeptide that the modified B cells are engineered to express. In some aspects, the sub-optimal single-dose concentration comprises ½ or 3, 4, 5, 6, 7, 8, 9, 10 fold, or less than the optimal single-dose concentration. In some aspects, the therapeutic polypeptide is IDUA.

In one embodiment, a single dose of modified B cells is administered to a subject. In one embodiment, two or more doses of modified B cells are administered sequentially to a subject. In one embodiment, three doses of modified B cells are administered sequentially to a subject. In one embodiment, a dose of modified B cells is administered weekly, biweekly, monthly, bimonthly, quarterly, semiannually, annually, or biannually to a subject. In one embodiment, a second or subsequent dose of modified B cells is administered to a subject when an amount of a therapeutic agent produced by the modified B cells decreases.

In some embodiments, lower numbers of the B cell, in the range of 10⁶/kilogram may be administered. In some embodiments, the B cells are administered at 1×10⁴, 5×10⁴, 1×10⁵, 5×10⁵, 1×10⁶, 5×10⁶, 1×10⁷, 5×10⁷, 1×10⁸, 5×10⁸, 5×10⁹, 1×10¹⁰, 5×10¹⁰, 1×10¹¹, 5×10¹¹, or 1×10¹² cells to the subject.

In some embodiments, a dose of modified B cells is administered to a subject at a certain frequency (e.g., weekly, biweekly, monthly, bimonthly, or quarterly) until a desired amount (e.g., an effective amount) of a therapeutic agent is detected in the subject. In some embodiments, an amount of the therapeutic agent is monitored in the subject. In one embodiment, a subsequent dose of modified B cells is administered to the subject when the amount of the therapeutic agent produced by the modified B cells decreases below the desired amount. In some embodiments, the desired amount is a range that produces the desired effect. For example, in a method for reducing the amount of glycosaminoglycans (GAGs) in an individual with MPS I, a desired amount of IDUA is an amount that decreases the level of GAGs in a certain tissue in comparison to the level of GAGs in the absence of IDUA.

The B cells of the present disclosure may also be administered using any number of matrices. Matrices have been utilized for a number of years within the context of tissue engineering (see, e.g., Principles of Tissue Engineering (Lanza, Langer, and Chick (eds.), 1997). The present disclosure utilizes such matrices within the novel context of acting as an artificial lymphoid organ to support and maintain the B cells. Accordingly, the present disclosure can utilize those matrix compositions and formulations which have demonstrated utility in tissue engineering. Accordingly, the type of matrix that may be used in the compositions, devices and methods of the disclosure is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized. Matrices comprise features commonly associated with being biocompatible when administered to a mammalian host. Matrices may be formed from natural and/or synthetic materials. The matrices may be nonbiodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present disclosure is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current disclosure is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In some embodiments, the subject is a mammal. In some embodiments, the subject is a human

In some embodiments, the subject has a disease or disorder associated with lysosomal storage dysfunction. In some embodiments, the disease or disorder associated with lysosomal storage dysfunction is caused by enzyme alpha-L-iduronidase (IDUA) deficiency. In some embodiments, the subject has mucopolysaccharidosis type I (MPS I). In some embodiments, the administration of the genetically modified B cells treats the subject's disease or disorder associated with lysosomal storage dysfunction. In some embodiments, the administration of the genetically modified B cells treats the subject's MPS I.

In some embodiments, the administering a B cell genetically modified to express IDUA (IDUA+ B cells) is used to treat a subject having, or suspected of having, MPS I. In some embodiments, a single, optimal dose of IDUA+ B cells is administered to the subject. In some embodiments, two or more doses of IDUA+ B cells are administered to the subject. In some embodiments, the two or more doses of IDUA+ B cells that are administered to the subject comprise less IDUA+ B cells than the single, optimal dose of IDUA+ B cells. In some embodiments, when two or more doses of IDUA+ B cells are administered to a subject at a dosage of IDUA+ B cells that is below the maximally effective single dose of IDUA+ B cells. In some embodiments, administering IDUA+ B cells to a subject results in normal levels of IDUA seen in a healthy, control subject. In some embodiments, administering IDUA+ B cells to a subject results in greater than normal levels of IDUA in the subject. In some embodiments, administering IDUA+ B cells to a subject reduces levels of GAGs in the subject to a normal level. In some embodiments, administering IDUA+ B cells to a subject reduces levels of GAGs in the subject to less than a normal level of GAGs in the subject.

### Therapeutic Agent

As used herein “gene of interest” or “gene” or “nucleic acid of interest” refers to a transgene to be expressed in the target transfected cell. While the term “gene” may be used, this is not to imply that this is a gene as found in genomic DNA and is used interchangeably with the term “nucleic acid”. Generally, the nucleic acid of interest provides suitable nucleic acid for encoding a therapeutic agent and may comprise cDNA or DNA and may or may not include introns, but generally does not include introns. As noted elsewhere, the nucleic acid of interest is operably linked to expression control sequences to effectively express the protein of interest in the target cell. In some embodiments, the vectors described herein may comprise one or more genes of interest, and may include 2, 3, 4, or 5 or more genes of interest.

A therapeutic agent to be delivered by a genetically modified B cell as described herein may be a protein. A protein of interest for use as described herein comprises any protein providing an activity desired. In this regard, a protein of interest includes, but is not limited to, an enzyme.

In some embodiments, the nucleic acid of interest encodes a protein. In some embodiments, the nucleic acid of interest encodes an enzyme. In some embodiments, the nucleic acid of interest encodes an enzyme to treat a lysosomal storage disorder. In some embodiments, the nucleic acid of interest encodes iduronidase (IDUA).

In some embodiments, the therapeutic agent produced by the modified B cell is a protein. In some embodiments, the therapeutic agent produced by the genetically modified B cell is an enzyme. In some embodiments, the therapeutic agent produced by the genetically modified B cells is iduronidase (IDUA).

Thus, this disclosure provides polynucleotides (isolated or purified or pure polynucleotides) encoding therapeutic agents (e.g., proteins of interest) of this disclosure for genetically modifying B cells, vectors (including cloning vectors and expression vectors) comprising such polynucleotides, and cells (e.g., host cells) transformed or transfected with a polynucleotide or vector according to this disclosure. In certain embodiments, a polynucleotide (DNA or RNA) encoding a protein of interest of this disclosure is contemplated. Expression cassettes encoding proteins of interest are also contemplated herein.

The present disclosure also relates to vectors that include a polynucleotide of this disclosure and, in particular, to recombinant expression constructs. In one embodiment, this disclosure contemplates a vector comprising a polynucleotide encoding a protein of this disclosure, along with other polynucleotide sequences that cause or facilitate transcription, translation, and processing of such a protein-encoding sequences. Appropriate cloning and expression vectors for use with prokaryotic and eukaryotic hosts are described, for example, in Sambrook et al, Molecular Cloning: A Laboratory Manual, Second Edition, Cold Spring Harbor, NY, (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein.

### Cells and Compositions

In some embodiments, the modified B cells described herein have been activated/differentiated in vitro and transfected to express a therapeutic agent as described herein. In some embodiments, the modified B cells described herein have been activated/differentiated in vitro and engineered (e.g., using a targeted transgene integration approach such as a zinc finger nuclease, TALEN, meganuclease, or CRISPR-mediated transgene integration) to express a therapeutic agent as described herein. In some embodiments, the compositions comprise B cells that have differentiated into plasma B cells, have been transfected or otherwise engineered and express one or more proteins of interest. Target cell populations, such as the transfected or otherwise engineered and activated B cell populations of the present disclosure may be administered either alone, or as a pharmaceutical composition in combination with diluents and/or with other components such as cytokines or cell populations.

In one embodiment, the modified B cells that have been engineered to express one or more proteins of interest are harvested from culture after activation/differentiation in vitro at a time-point at which the modified B cells have optimal migratory capacity for a particular chemoattractant. In some embodiments, the optimal migratory capacity may be on day 7, day 8, or day 9 of the B cell culture. In some embodiments, the optimal migratory capacity may be on day 5, day 6, or day 7 of the B cell culture after transfection or engineering. In some embodiments, the optimal migratory capacity may be on day 8 of the B cell culture after transfection or engineering or later in culture than day 8 after transfection or engineering (e.g., day 9, 10, 11, 12, 13, 14, 15, 16, 17, 18, 19, 20, or later than day 20). In some embodiments, the optimal migratory capacity may be prior to day 10 of the B cell culture. In some embodiments, the optimal migratory capacity may be prior to day 8 of the B cell culture after transfection or engineering. In some embodiments, the optimal migratory capacity may be on day 6 or day 7 of the B cell culture. In some embodiments, the optimal migratory capacity may be on day 4 or day 5 of the B cell culture after transfection or engineering. In some embodiments, the optimal migratory capacity may be prior to day 9 of the B cell culture. In some embodiments, the optimal migratory capacity may be prior to day 7 of the B cell culture after transfection or engineering. In some embodiments, the optimal migratory capacity is optimal for modified B cell homing to CXCL12. In some embodiments, the optimal migratory capacity is optimal for modified B cell homing to the bone marrow of a subject receiving one or more administration of the modified B cells. In some embodiments, the B cells are harvested for administration to a subject at optimal migratory capacity to CXCL12 and/or to the bone marrow of a subject on from about day 7 to about day 9 in culture. In some embodiments, the B cells are harvested for administration to a subject at optimal migratory capacity to CXCL12 and/or to the bone marrow of a subject on from about day 5 to about day 7 in culture after transfection or engineering. In some embodiments, the B cells are harvested for administration to a subject at optimal migratory capacity to CXCL12 and/or to the bone marrow of a subject prior to about day 10 in culture. In some embodiments, the B cells are harvested for administration to a subject at optimal migratory capacity to CXCL12 and/or to the bone marrow of a subject prior to about day 8 in culture after transfection or engineering. In some embodiments, the optimal migratory capacity is optimal for modified B cell homing to CXCL13. In some embodiments, the optimal migratory capacity is optimal for modified B cell homing to a site of inflammation in a subject receiving one or more administration of the modified B cells. In some embodiments, the B cells are harvested for administration to a subject at optimal migratory capacity to CXCL13 and/or to a site of inflammation in the subject on about day 6 or about day 7 in culture. In some embodiments, the B cells are harvested for administration to a subject at optimal migratory capacity to CXCL13 and/or to a site of inflammation in the subject on about day 4 or about day 5 in culture after transfection or engineering. In some embodiments, the B cells are harvested for administration to a subject at optimal migratory capacity to CXCL13 and/or to a site of inflammation prior to about day 10 in culture. In some embodiments, the B cells are harvested for administration to a subject at optimal migratory capacity to CXCL13 and/or to a site of inflammation prior to about day 8 in culture after transfection or engineering.

In some embodiments, the optimal migratory capacity is optimal for modified B cell homing to both CXCL12 and CXCL13. In some embodiments, the B cells are harvested at optimal migratory capacity for homing to both CXCL12 and CXCL13 on day 7 of the B cell culture. In some embodiments, the B cells are harvested at optimal migratory capacity for homing to both CXCL12 and CXCL13 on day 5 of the B cell culture after transfection or engineering.

In some embodiments, the engineered B cells are harvested when at least about 20% of the B cells migrate in a chemotaxis assay to a particular chemoattractant. For example, but not to be limited by example, the engineered B cells (e.g., that produce IDUA) may be harvested when at least about 20% of the B cells migrate in a chemotaxis assay to CXCL12. Or, in another non-limiting example, the engineered B cells (e.g., that produce IDUA) may be harvested when at least about 20% of the B cells migrate in a chemotaxis assay to CXCL13. Furthermore, the engineered B cells (e.g., that produce IDUA) may be harvested when at least about 30% of the B cells migrate in a chemotaxis assay to a particular chemoattractant (e.g., CXCL12 or CXCL13), or when at least about 40%, 45%, 50%, 55%, 60%, 65%, or at least about 70% of the B cells migrate in a chemotaxis assay to a particular chemoattractant (e.g., CXCL12 or CXCL13). Furthermore, the engineered B cells (e.g., that produce IDUA) may be harvested when more than 70% of the B cells migrate in a chemotaxis assay. Such chemotaxis assays are known in the art and are described herein (see, e.g., Example 6 herein).

Briefly, cell compositions of the present disclosure may comprise a differentiated and activated B cell population that has been transfected and is expressing a therapeutic agent as described herein, in combination with one or more pharmaceutically or physiologically acceptable carriers, diluents or excipients. Such compositions may comprise buffers such as neutral buffered saline, phosphate buffered saline, Lactated Ringer's solution and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for direct injection into the central nervous system.

In some embodiments, the modified B cells may comprise a pharmaceutical composition.

As used herein the term “pharmaceutical composition” refers to a pharmaceutical acceptable composition, wherein the composition comprises engineered B cells, and in some embodiments further comprises a pharmaceutically acceptable carrier.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carriers” or “pharmaceutically effective excipients” refers to an excipient, diluent, preservative, solubilizer, emulsifier, adjuvant, and/or vehicle with which an engineered B cell, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solution s or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation.

In some embodiments, one or more pharmaceutically active ingredients may be used in combination with the engineered B cells.

In some embodiments, B cells transfected and activated using the methods described herein, or other methods known in the art, are administered to a patient in conjunction with (e.g. before, simultaneously or following) any number of relevant treatment modalities, including but not limited to treatment with agents such as antiviral agents, chemotherapy, radiation, immunosuppressive agents, such as cyclosporin, bisulfin, bortezomib, azathioprine, methotrexate, mycophenolate, and FK506, antibodies, or other immunoablative agents such as CAMPATH, anti-CD3 antibodies or other antibody therapies, cytoxin, fiudaribine, cyclosporin, FK506, rapamycin, mycophenolic acid, steroids, FR901228, cytokines, and irradiation. These drugs inhibit either the calcium dependent phosphatase calcineurin (cyclosporine and FK506), the proteasome (bortezomib), or inhibit the p70S6 kinase that is important for growth factor induced signaling (rapamycin). (Liu et al, Cell 66:807-815, 1991; Henderson et al, Immun. 73:316-321, 1991; Bierer et al, Curr. Opin. Immun. 5:763-773, 1993; Isoniemi (supra)).

The dosage of the above compositions to be administered to a patient will vary with the precise nature of the condition being treated and the recipient of the treatment. The scaling of dosages for human administration can be performed according to art-accepted practices.

In some embodiments, a cell composition is assessed for purity prior to administration. In some embodiments, a cell composition is tested for robustness of therapeutic agent production. In some embodiments, a cell composition is tested for sterility. In some embodiments, a cell composition is screened to confirm it matches the recipient subject.

In some embodiments, a cell composition is stored and/or shipped at 4° C. In another embodiment, a cell composition is frozen for storage and/or shipment. A cell composition may be frozen at, e.g., −20° C. or −80° C. In some embodiments, a step of freezing a cell composition comprises liquid nitrogen. In one embodiment, a cell composition is frozen using a controlled rate freezer. Accordingly, methods described herein may further include a thawing step.

### Engineered B Cells

In certain embodiments of the methods described herein, B cells can be obtained from a unit of blood collected from a subject using any number of techniques known to the skilled artisan, such as FICOLL™ (copolymers of sucrose and epichlorohydrin that may be used to prepare high density solutions) separation. B cells can be obtained from a number of sources, including peripheral blood mononuclear cells (PBMCs), bone marrow, lymph node tissue, cord blood, tissues from a site of infection, spleen tissue, and tumors. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In one embodiment of the methods described herein, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, PBS. Alternatively, the undesirable components of the apheresis sample may be removed and the cells directly resuspended in culture media.

B cells may be isolated from peripheral blood or leukapheresis using techniques known in the art. For example, PBMCs may be isolated using FICOLL™ (Sigma-Aldrich, St Louis, MO) and CD19+ B cells purified by negative or positive selection using any of a variety of antibodies known in the art, such as the Rosette tetrameric complex system (StemCell Technologies, Vancouver, Canada) or MACS™ MicroBead Technology (Miltenyi Biotec, San Diego, CA). In some embodiments, memory B cells are isolated as described by Jourdan et al, (Blood. 2009 Dec. 10; 114(25):5173-81). For example, after removal of CD2+ cells using anti-CD2 magnetic beads, CD19+ CD27+ memory B cells can be sorted by FACS. Bone marrow plasma cells (BMPCs) can be purified using anti-CD138 magnetic microbeads sorting or other similar methods and reagents. Human B cells may be isolated, e.g., using CD19 MicroBeads, human (Miltenyi Biotec, San Diego, CA). Human Memory B cell may be isolated, e.g., using the Memory B Cell Isolation Kit, human (Miltenyi Biotec, San Diego, CA).

Other isolation kits are commercially available, such as R&D Systems' MagCellect Human B Cell Isolation Kit (Minneapolis, MN). In some embodiments, resting B cells may be prepared by sedimentation on discontinuous Percoll gradients, as described in (Defranco et al, (1982) J. Exp. Med. 155: 1523).

In some embodiments, peripheral blood mononuclear cells (PBMCs) are obtained from a blood sample using a gradient based purification (e.g., FICOLL™). In some embodiments, PBMCs are obtained from apheresis based collection. In one embodiment, B cells are isolated from PBMCs by isolating pan B cells. The isolating step may utilize positive and/or negative selection. In some embodiments, the negative selection comprises depleting T cells using anti-CD3 conjugated microbeads, thereby providing a T cell depleted fraction. In some embodiments, memory B cells are isolated from the pan B cells or the T cell depleted fraction by positive selection for CD27. In one particular embodiment, memory B cells are isolated by depletion of unwanted cells and subsequent positive selection with CD27 MicroBeads. Unwanted cells, for example, T cells, NK cells, monocytes, dendritic cells, granulocytes, platelets, and erythroid cells may be depleted using a cocktail of biotinylated antibodies against CD2, CD14, CD16, CD36, CD43, and CD235a (glycophorin A), and Anti-Biotin MicroBeads.

In some embodiments, switched memory B cells are obtained. “Switched memory B cell” or “switched B cell,” as used herein, refers to a B cell that has undergone isotype class switching. In some embodiments, switched memory B cells are positively selected for IgG. In some embodiments, switched memory B cells are obtained by depleting IgD and IgM expressing cells. Switched memory B cells may be isolated, e.g., using the Switched Memory B Cell Kit, human (Miltenyi Biotec, San Diego, CA).

For example, in some embodiments, non-target cells may be labeled with a cocktail of biotinylated CD2, CD14, CD16, CD36, CD43, CD235a (glycophorin A), Anti-IgM, and Anti-IgD antibodies. These cells may be subsequently magnetically labeled with Anti-Biotin MicroBeads. Highly pure switched memory B cells may be obtained by depletion of the magnetically labeled cells.

In some embodiments, the promoter sequence from a gene unique to memory B cells, such as, e.g., the CD27 gene (or other gene specific to memory B cells and not expressed in naive B cells) is used to drive expression of a selectable marker such as, e.g., mutated dihydrofolate reductase allowing for positive selection of the memory B cells in the presence of methotrexate. In another embodiment, the promoter sequence from a pan B cell gene such as, e.g., the CD19 gene is used to drive expression of a selectable marker such as, e.g., mutated dihydrofolate reductase allowing for positive selection of the memory B cells in the presence of methotrexate. In another embodiment T cells are depleted using CD3 or by addition of cyclosporin. In some embodiments, CD138+ cells are isolated from the pan B cells by positive selection. In some embodiments, CD138+ cells are isolated from PBMCs by positive selection. In some embodiments, CD38+ cells are isolated from the pan B cells by positive selection. In some embodiments, CD38+ cells are isolated from PBMCs by positive selection. In some embodiments, CD27+ cells are isolated from PBMCs by positive selection. In some embodiments, memory B cells and/or plasma cells are selectively expanded from PBMCs using in vitro culture methods available in the art.

In some embodiments, the genetically modified B cells are autologous to the subject. In some embodiments, the genetically modified B cells are allogeneic to the subject.

B cells, such as memory B cells, can be cultured using in vitro methods to activate and differentiate the B cells into plasma cells or plasmablasts or both. As would be recognized by the skilled person, plasma cells may be identified by cell surface protein expression patterns using standard flow cytometry methods. For example, terminally differentiated plasma cells express relatively few surface antigens, and do not express common pan-B cell markers, such as CD19 and CD20. Instead, plasma cells may be identified by expression of CD38, CD78, CD138, and IL-6R and lack of expression of CD45. CD27 may also be used to identify plasma cells as naive B cells are CD27−, memory B cells are CD27+ and plasma cells are CD27++. Plasma cells express high levels of CD38 and CD138.

In some embodiments, the genetically modified B cells are CD20−, CD38+, and CD138+. In some embodiments, the genetically modified B cells are CD20−, CD38+, and CD138−.

As used herein, unless as otherwise described with regard to viral vectors, “vector” means a nucleic acid molecule capable of transporting another nucleic acid to which it has been linked. Exemplary vectors include plasmids, minicircles, transposons (e.g., Sleeping Beauty transposon), yeast artificial chromosomes, self-replicating RNAs, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences. In certain embodiments, expression constructs are derived from plasmid vectors. Illustrative constructs include modified pNASS vector (Clontech, Palo Alto, CA), which has nucleic acid sequences encoding an ampicillin resistance gene, a polyadenylation signal and a T7 promoter site; pDEF38 and pNEF38 (CMC ICOS Biologies, Inc.), which have a CHEF1 promoter; and pD18 (Lonza), which has a CMV promoter. Other suitable mammalian expression vectors are well known (see, e.g., Ausubel et al, 1995; Sambrook et al, supra; see also, e.g., catalogs from Invitrogen, San Diego, CA; Novagen, Madison, WI; Pharmacia, Piscataway, NJ).

Useful constructs may be prepared that include a dihydrofolate reductase (DHFR)-encoding sequence under suitable regulatory control, for promoting enhanced production levels of the fusion proteins, which levels result from gene amplification following application of an appropriate selection agent (e.g., methotrexate). In one embodiment, use of a bifunctional transposon encoding a therapeutic gene (e.g., IDUA) along with drug-resistant DHFR in combination with incubation in methotrexate (MTX) to enrich for successfully transposed B cells, generates a more potent product.

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, encoding nucleic acids as provided herein may be included in any one of a variety of expression vector constructs (e.g., minicircles) as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (Current Protocols in Molecular Biology, Greene Publ. Assoc. Inc. & John Wiley & Sons, Inc., Boston, MA, 1993); Sambrook et al. (Molecular Cloning, Second Ed., Cold Spring Harbor Laboratory, Plainview, NY, 1989); Maniatis et al. (Molecular Cloning, Cold Spring Harbor Laboratory, Plainview, NY, 1982); Glover (Ed.) (DNA Cloning Vol. I and II, IRL Press, Oxford, UK, 1985); Hames and Higgins (Eds.) (Nucleic Acid Hybridization, IRL Press, Oxford, UK, 1985); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors, kanamycin vectors, or other vectors with selectable markers. Eukaryotic promoters include CMV immediate early, HSV thymidine kinase, early and late SV40, LTRs from retrovirus, EEK, EF1alpha, and mouse metallothionein-1. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

In some embodiments, the plasmid may comprise a sequence of SEQ ID NO: 1. In some embodiments, the plasmid may consist of a sequence of SEQ ID NO: 1. In some embodiments, the plasmid may comprise or consist of a sequence that is at least about 60% identical to SEQ ID NO: 1. In some embodiments, the plasmid may comprise or consist of a sequence that is at least about 85% identical to SEQ ID NO: 1, or at least about 90%, 95%, 96%, 97%, 98%, 99%, or greater than 99% identical to SEQ ID NO: 1.

Variants of the polynucleotides of this disclosure are also contemplated. Variant polynucleotides are at least 60%, 65%, 70%, 75%, 80%, 85%, 90%, and preferably 95%, 96%, 97%, 98%, 99%, or 99.9% identical to one of the polynucleotides of defined sequence as described herein, or that hybridizes to one of those polynucleotides of defined sequence under stringent hybridization conditions of 0.015M sodium chloride, 0.0015M sodium citrate at about 65-65° C. or 0.015M sodium chloride, 0.0015M sodium citrate, and 50% formamide at about 42° C. The polynucleotide variants retain the capacity to encode a binding domain or fusion protein thereof having the functionality described herein.

In some embodiments, the genetically modified B cells are transfected with a transgene. Exemplary methods for transfecting B cells are provided in WO 2014/152832 and WO 2016/100932, both of which are incorporated herein by reference in their entireties. Transfection of B cells may be accomplished using any of a variety of methods available in the art to introduce DNA or RNA into a B cell. Suitable techniques may include calcium phosphate transfection, DEAE-Dextran, electroporation, pressure-mediated transfection or “cell squeezing” (e.g., CellSqueeze microfluidic system, SQZ Biotechnologies), nano-particle-mediated or liposome-mediated transfection and transduction using retrovirus or other virus, e.g., vaccinia. See, e.g., Graham et al., 1973, Virology 52:456; Sambrook et al, 2001, Molecular Cloning, a Laboratory Manual, Cold Spring Harbor Laboratories; Davis et al, 1986, Basic Methods in Molecular Biology, Elsevier; Chu et al, 1981, Gene 13: 197; U.S. Pat. Nos. 5,124,259; 5,297,983; 5,283,185; 5,661,018; 6,878,548; 7,799,555; 8,551,780; and 8,633,029. One example of a commercially available electroporation technique suitable for B cells is the Nucleofector™ transfection technology. Transfection may take place prior to or during in vitro culture of the isolated B cells in the presence of one or more activating and/or differentiating factors described above. For example, cells are transfected on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of in vitro culture. In some embodiments, cells are transfected on day 1, 2, or 3 of in vitro culture. In some embodiments, cells are transfected on day 2. For example, cells are electroporated on day 2 of in vitro culture for delivery of, e.g., a plasmid, a transposon, a minicircle, or a self-replicating RNA. In another embodiment, cells are transfected on day 4, 5, 6, or 7 of in vitro culture. In some embodiments, cells are transfected on day 6 of in vitro culture. In another embodiment, cells are transfected on day 5 of in vitro culture.

In some embodiments, cells are transfected or otherwise engineered (e.g., via a targeted integration of a transgene) prior to activation. In some embodiments, cells are transfected or otherwise engineered (e.g., via a targeted integration of a transgene) during activation. In some embodiments, cells are transfected or otherwise engineered (e.g., via a targeted integration of a transgene) after activation. In some embodiments, cells are transfected or otherwise engineered (e.g., via a targeted integration of a transgene) prior to differentiation. In some embodiments, cells are transfected or otherwise engineered (e.g., via a targeted integration of a transgene) during differentiation. In some embodiments, cells are transfected or otherwise engineered (e.g., via a targeted integration of a transgene) after differentiation.

In some embodiments, a non-viral vector is used to deliver DNA or RNA to memory B cells and/or plasma cells. For example, systems that may facilitate transfection of memory B cells and/or plasma cells without the need of a viral integration system include, without limitation, transposons (e.g., Sleeping Beauty transposon system), zinc-finger nucleases (ZFNs), transcription activator-like effector nucleases (TALENs), clustered regularly interspaced short palindromic repeats (CRISPRs), meganucleases, minicircles, replicons, artificial chromosomes (e.g., bacterial artificial chromosomes, mammalian artificial chromosomes, and yeast artificial chromosomes), plasmids, cosmids, and bacteriophage.

In some embodiments, the genetically modified B cells were prepared using a Sleeping Beauty transposon to express the therapeutic agent in the B cells. In some embodiments, the genetically modified B cells were prepared using a recombinant viral vector to express the therapeutic agent in the B cells. In some embodiments, the recombinant viral vector encodes a recombinant retrovirus, recombinant lentivirus, recombinant adenovirus, or recombinant adeno-associated virus.

In some embodiments, such non-viral-dependent vector systems may also be delivered via a viral vector known in the art or described below. For example, in some embodiments, a viral vector (e.g., a retrovirus, lentivirus, adenovirus, adeno-associated virus), is utilized to deliver one or more non-viral vector (such as, e.g., one or more of the above-mentioned zinc-finger nucleases (ZFNs), transcription activator-like effector nucleases (TALENs), clustered regularly interspaced short palindromic repeats (CRISPRs) meganucleases, or any other enzyme/complementary vectors, polynucleotides, and/or polypeptides capable of facilitating the targeted integration. Accordingly, in some embodiments, a cell (e.g., B cells such as a memory B cells and/or plasma cells) may be engineered to express an exogenous sequence (e.g., a sequence encoding a therapeutic polypeptide such as IDUA) via a targeted integration method. Such methods are known in the art and may comprise cleaving an endogenous locus in the cell using one or more nucleases (e.g., ZFNs, TALENs, CRISPR/Cas, meganucleases) and administering the transgene to the cell such that it is integrated into the endogenous locus and expressed in the cell. The transgene may be comprised in a donor sequence that is integrated into the host cell's DNA at or near the point of a cleavage by the nuclease. In some embodiments, the genetically modified B cells were prepared by gene editing of the B cell genome or by targeted integration into the genome of the B cell of a polynucleotide sequence encoding the therapeutic agent.

In some embodiments, the targeted integration comprises a zinc finger nuclease-mediated gene integration, CRISPR-mediated gene integration or gene editing, TALE-nuclease-mediated gene integration, or meganuclease-mediated gene integration. In some embodiments, the targeted integration of polynucleotide occurred via homologous recombination. In some embodiments, the targeted integration comprises a viral vector-mediated delivery of a nuclease capable of inducing a DNA cleavage at a target site. In some embodiments, the nuclease is a zinc finger nuclease, a Cas nuclease, a TALE-nuclease, or a meganuclease.

The integration of the exogenous sequence (e.g., a sequence encoding a therapeutic polypeptide such as IDUA) may occur via recombination. As would be clear to one of skill in the art, “Recombination” refers to a process of exchange of genetic information between two polynucleotides, including but not limited to, donor capture by non-homologous end joining (NHEJ) and homologous recombination. The recombination may be homologous recombination. For the purposes of this disclosure, “homologous recombination (HR)” refers to the specialized form of such exchange that takes place, for example, during repair of double-strand breaks in cells via homology-directed repair mechanisms. This process utilizes nucleotide sequence homology, whereby a “donor” molecule (e.g., donor polynucleotide sequence or donor vector comprising such a sequence) is utilized by a cell's DNA-repair machinery as a template to repair of a “target” molecule (i.e., the one that experienced the double-strand break), and by these means causes the transfer of genetic information from the donor to the target. In some embodiments of HR-directed integration, the donor molecule may contain at least 2 regions of homology to the genome (“homology arms”). In some embodiments, the homology arms may be, e.g., of least 50-100 base pairs in length. The homology arms may have substantial DNA homology to a region of genomic DNA flanking the cleavage site wherein the targeted integration is to occur. The homology arms of the donor molecule may flank the DNA that is to be integrated into the target genome or target DNA locus. Breakage of the chromosome followed by repair using the homologous region of the plasmid DNA as a template may results in the transfer of the intervening transgene flanked by the homology arms into the genome. See, e.g., Roller et al. (1989) Proc. Natl. Acad Sci. USA. 86(22): 8927-8931; Thomas et al. (1986) Cell 44(3):419-428. The frequency of this type of homology-directed targeted integration can be increased by up to a factor of 105 by deliberate creation of a double-strand break in the vicinity of the target region (Hockemeyer et al. (2009) Nature Biotech. 27(9):851-857; Lombardo et al. (2007) Nature Biotech. 25(11): 1298-1306; Moehle et al. (2007) Proc. Natl. Acad. Sci. USA 104(9):3055-3060: Rouet et al. (1994) Proc. Natl. Acad. Sci. USA 91 (13):6064-6068.

Any nuclease capable of mediating the targeted cleavage of a genomic locus such that a trans gene may be integrated into the genome of a target cell (e.g., by recombination such as HR) may be utilized in engineering a cell (e.g., a memory B cell or plasmablast) according to the present disclosure.

A double-strand break (DSB) or nick can be created by a site-specific nuclease such as a zinc-finger nuclease (ZFN), a TAL effector domain nuclease (TALEN). a meganuclease, or using the CRISPR-mediated system with an engineered crRNA/tract RNA (single guide RNA) to guide specific cleavage. See. for example, Burgess (2013) Nature Reviews Genetics 14:80-81, Umov et al. (2010) Nature 435(7042):646-51; U.S. Pat. Pub. Nos. 2003/0232410; 2005/0208489; 2005/0026157, 2005/0064474; 2006/0188987, 2009/0263900; 2009/0117617; 2010/0047805; 2011/0207221; 2011/0301073 and Int'l Pat. Pub. No. WO 2007/014275, the disclosures of which are incorporated by reference in their entireties for all purposes.

In some embodiments, the cell (e.g., a memory B cell or a plasmablast) is engineered via Zinc Finger Nuclease-mediated targeted integration of a donor construct. A zinc finger nuclease (ZFN) is an enzyme that is able to recognize and cleave a target nucleotide sequence with specificity due to the coupling of a “zinc finger DNA binding protein” (ZFP) (or binding domain), which binds DNA in a sequence-specific manner through one or more zinc fingers, and a nuclease enzyme. ZFNs may comprise any suitable cleavage domains (e.g., a nuclease enzyme) operatively linked to a ZFP DNA-binding domain to form a engineered ZFN that can facilitate site-specific cleavage of a target DNA sequence (see, e.g., Kim et al. (1996) Proc Natl Acad Sci USA 93(3): 1156-1160). For example, ZFNs may comprise a target-specific ZFP linked to a FORI enzyme or a portion of a FOK1 enzyme. In some embodiments, ZFN used in a ZFN-mediated targeted integration approach utilize two separate molecules, each comprising a subunit of a FOK1 enzyme each bound to a ZFP, each ZFP with specificity for a DNA sequence flanking a target cleavage site, and when the two ZFPs bind to their respective target DNA sites the FOK1 enzyme subunits are brought into proximity with one another and they bind together activating the nuclease activity which cleaves the target cleavage site. ZFNs have been used for genome modification in a variety of organisms (e.g., United States Patent Publications 20030232410; 20050208489; 20050026157; 20050064474; 20060188987; 20060063231; and International Publication WO 07/014,275, incorporated herein by reference in their entirety) Custom ZFPs and ZFNs are commercially available from, e.g., Sigma Aldrich (St. Louis, MO), and any location of DNA may be routinely targeted and cleaved using such custom ZFNs.

In some embodiments, the cell (e.g., a memory B cell or a plasmablast) is engineered via CRISPR-mediated (e.g., CRISPR/Cas) Nuclease-mediated integration of a donor construct. A CRISPR (Clustered Regularly Interspaced Short Palindromic Repeats)/Cas (CRISPR Associated) nuclease system is an engineered nuclease system based on a bacterial system that may be used for genome engineering. It is based on part of the adaptive immune response of many bacteria and archea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas (e.g. Cas9) nuclease to a region homologous to the crRNA in the target DNA called a “protospacer”. Cas cleaves the DNA to generate blunt ends at the DSB at sites specified by a 20-nucleotide guide sequence contained within the crRNA transcript. Cas requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas nuclease to target any desired sequence (see Jinek et al (2012) Science 337, p. 816-821, Jinek et al, (2013), eLife 2:e00471, and David Segal, (2013) eLife 2:e00563). Thus, the CRISPR/Cas system can be engineered to create a DSB at a desired target in a genome, and repair of the DSB can be influenced by the use of repair inhibitors to cause an increase in error prone repair. In some embodiments, the CRISPR/Cas nuclease-mediated integration utilizes a Type II CRISPR. The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas to the target DNA via Watson-Crick base-pairing between the spacer on the crRNA and the protospacer on the target DNA next to a protospacer adjacent motif (PAM), an additional requirement for target recognition. Forth, Cas mediates cleavage of target DNA to create a double-stranded break within the protospacer.

The Cas related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al, (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas) to create a chimeric cr-RNA-tracrRNA hybrid (also termed a single guide RNA). (see Jinek ibid and Cong, ibid).

In some embodiments, a single guide RNA containing both the crRNA and tracrRNA may be engineered to guide the Cas nuclease to target any desired sequence (e.g., Jinek et al (2012) Science 337, p. 816-821, Jinek et al, (2013), eLife 2:e00471, David Segal, (2013) eLife 2:e00563). Thus, the CRISPR/Cas system may be engineered to create a DSB at a desired target in a genome.

Custom CRISPR/Cas systems are commercially available from, e.g., Dharmacon (Lafayette, CO), and any location of DNA may be routinely targeted and cleaved using such custom single guide RNA sequences. Single stranded DNA templates for recombination may be synthesized (e.g., via oligonucleotide synthesis methods known in the art and commercially available) or provided in a vector, e.g., a viral vector such as an AAV. In some embodiments, the cell (e.g., a memory B cell or a plasmablast) is engineered via TALE-Nuclease (TALEN) mediated targeted integration of a donor construct. A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TAL-effectors may contain a nuclear localization sequence, an acidic transcriptional activation domain and a centralized domain of tandem repeats where each repeat contains approximately 34 amino acids that are key to the DNA binding specificity of these proteins, (e.g., Schornack S, et al (2006) J Plant Physiol 163(3): 256-272). TAL effectors depend on the sequences found in the tandem repeats which comprises approximately 102 bp and the repeats are typically 91-100% homologous with each other (e.g., Bonas et al (1989) Mol Gen Genet 218: 127-136). These DNA binding repeats may be engineered into proteins with new combinations and numbers of repeats, to make artificial transcription factors that are able to interact with new sequences and activate the expression of a non-endogenous reporter gene (e.g., Bonas et al (1989) Mol Gen Genet 218: 127-136). Engineered TAL proteins may be linked to a Fokl cleavage half domain to yield a TAL effector domain nuclease fusion (TALEN) to cleave target specific DNA sequence (e.g., Christian et al (2010) Genetics epub 10.1534/genetics. l l0.120717).

Custom TALEN are commercially available from, e.g., Thermo Fisher Scientific (Waltham, MA), and any location of DNA may be routinely targeted and cleaved.

In some embodiments, the cell (e.g., a memory B cell or a plasmablast) is engineered via meganuclease-mediated targeted integration of a donor construct. A meganuclease (or “homing endonuclease”) is an endonuclease that binds and cleaves double-stranded DNA at a recognition sequence that is greater than 12 base pairs. Naturally occurring meganucleases may be monomelic (e.g., I-Scel) or dimeric (e.g., I-Crel). Naturally occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-Scel, I-Ceul, PI-PspI, PI-Sce, I-SceIV, I-Csml, I-Panl, I-Scell, I-Ppol, I-SceIII, I-Crel, I-Tevl, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82: 115-118; Perler et al. (1994) Nucleic Acids Res. 22, 1125-1127; Jasin (1996) Trends Genet. 12:224-228; Gimble et al. (1996) J. Mol. Biol. 263: 163-180; Argast et al. (1998) J. Mol. Biol. 280:345-353 and the New England Biolabs catalogue. The term “Meganuclease” includes monomeric meganucleases, dimeric meganucleases and monomers that associate to form dimeric meganucleases.

In some embodiments, the methods and compositions described herein make use of a nuclease that comprises an engineered (non-naturally occurring) homing endonuclease (meganuclease). The recognition sequences of homing endonucleases and meganucleases such as I-Scel, I-Ceul, PI-PspI, PI-Sce, I-SceIV, I-Csml, I-Panl, I-SceII, I-Ppol, I-SceIII, I-Crel, I-Tevl, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82: 115-118; Perler et al. (1994) Nucleic Acids Res. 22, 1125-1127; Jasin (1996) Trends Genet. 12:224-228; Gimble et al. (1996) J. Mol. Biol. 263: 163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31 :2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain. Custom meganuclease are commercially available from, e.g., New England Biolabs (Ipswich, MA), and any location of DNA may be routinely targeted and cleaved.

The engineering of the B cell may comprise administering one or more nucleases (e.g., ZFNs, TALENs, CRISPR/Cas, meganuclease) to a B cell, e.g., via one or more vectors encoding the nucleases, such that the vectors comprising the encoded nucleases are taken up by the B cell. The vectors may be viral vectors.

In some embodiments, the nucleases cleave a specific endogenous locus (e.g. safe harbor gene or locus of interest) in the cell (e.g., memory B cell or plasma cell) and one or more exogenous (donor) sequences (e.g., transgenes) are administered (e.g. one or more vectors comprising these exogenous sequences). The nuclease may induce a double-stranded (DSB) or single-stranded break (nick) in the target DNA. In some embodiments, targeted insertion of a donor transgene may be performed via homology directed repair (HDR), non-homology repair mechanisms (e.g., NHEJ-mediated end capture), or insertions and/or deletion of nucleotides (e.g. endogenous sequence) at the site of integration of a transgene into the cell's genome.

In some embodiments, a method of transfecting a B cell comprises electroporating the B cell prior to contacting the B cell with a vector. In some embodiments, cells are electroporated on day 1, 2, 3, 4, 5, 6, 7, 8, or 9 of in vitro culture. In some embodiments, cells are electroporated on day 2 of in vitro culture for delivery of a plasmid. In some embodiments, cells are transfected using a transposon on day 1, 2, 3, 4, 5, 6, 7, 8, or 9 of in vitro culture. In some embodiments, cells are transfected using a minicircle on day 1, 2, 3, 4, 5, 6, 7, 8, or 9 of in vitro culture. In some embodiments, electroporation of a Sleeping Beauty transposon takes place on day 2 of in vitro culture.

In some embodiments, the B cells are contacted with a vector comprising a nucleic acid of interest operably linked to a promoter, under conditions sufficient to transfect at least a portion of the B cells. In some embodiments the B cells are contacted with a vector comprising a nucleic acid of interest operably linked to a promoter, under conditions sufficient to transfect at least 5% of the B cells. In some embodiments, the B cells are contacted with a vector under conditions sufficient to transfect at least 5%, 10%, 15%, 20%, 25%, 30%, 35%, 40%, 45%, 50%, 55%, 60%, 70%, 75%, 80%, 85%, 90%, 95%, 96%, 97%, 98%, 99% or even 100% of the B cells. In some embodiments, the B cells, cultured in vitro as described herein, are transfected, in which case the cultured B cells are contacted with a vector as described herein under conditions sufficient to transfect at least 5%, 10% 15%, 20%, 25%, 30%, 35%, 40%, 45%, 50%, 55%, 60%, 70%, 75%, 80%, 85%, 90%, 95%, 96%, 97%, 98%, 99% or even 100% of the B cells.

Viral vectors may be employed to transduce memory B cells and/or plasma cells. Examples of viral vectors include, without limitation, adenovirus-based vectors, adeno-associated virus (AAV)-based vectors, retroviral vectors, retroviral- adenoviral vectors, and vectors derived from herpes simplex viruses (HSVs), including amplicon vectors, replication-defective HSV and attenuated HSV (see, e.g., Krisky, Gene Ther. 5: 1517-30, 1998; Pfeifer, Annu. Rev. Genomics Hum. Genet. 2: 177-211, 2001, each of which is incorporated by reference in its entirety). In some embodiments, cells are transduced with a viral vector (e.g., a lentiviral vector) on day 1, 2, 3, 4, 5, 6, 7, 8, or 9 of in vitro culture. In some embodiments, cells are transduced with a viral vector on day 5 of in vitro culture. In some embodiments, the viral vector is a lentivirus. In some embodiments, cells are transduced with a measles virus pseudotyped lentivirus on day 1 of in vitro culture.

In some embodiments, B cells are transduced with retroviral vectors using any of a variety of known techniques in the art (see, e.g., Science 12 Apr. 1996 272: 263-267; Blood 2007, 99:2342-2350; Blood 2009, 1 13: 1422-1431; Blood 2009 Oct. 8; 1 14(15):3173-80; Blood. 2003; 101 (6):2167-2174; Current Protocols in Molecular Biology or Current Protocols in Immunology, John Wiley & Sons, New York, N.Y.(2009)). Additional description of viral transduction of B cells may be found in WO 2011/085247 and WO 2014/152832, each of which is herein incorporated by reference in its entirety.

For example, PBMCs, B- or T-lymphocytes from donors, and other B cell cancer cells such as B-CLLs may be isolated and cultured in IMDM medium or RPMI 1640 (GibcoBRL Invitrogen, Auckland, New Zealand) or other suitable medium as described herein, either serum-free or supplemented with serum (e.g., 5-10% FCS, human AB serum, and serum substitutes) and penicillin/streptomycin and/or other suitable supplements such as transferrin and/or insulin. In some embodiments, cells are seeded at 1×10⁵ cells in 48-well plates and concentrated vector added at various doses that may be routinely optimized by the skilled person using routine methodologies. In one embodiment, B cells are transferred to an MS5 cell monolayer in RPMI supplemented with 10% AB serum, 5% FCS, 50 ng/ml rhSCF, 10 ng/ml rhlL-15 and 5 ng/ml rhlL-2 and medium refreshed periodically as needed. As would be recognized by the skilled person, other suitable media and supplements may be used as desired.

Some embodiments relate to the use of retroviral vectors, or vectors derived from retroviruses. “Retroviruses” are enveloped RNA viruses that are capable of infecting animal cells, and that utilize the enzyme reverse transcriptase in the early stages of infection to generate a DNA copy from their RNA genome, which is then typically integrated into the host genome. Examples of retroviral vectors Moloney murine leukemia virus (MLV)-derived vectors, retroviral vectors based on a Murine Stem Cell Virus, which provides long-term stable expression in target cells such as hematopoietic precursor cells and their differentiated progeny (see, e.g., Hawley et al., PNAS USA 93: 10297-10302, 1996; Keller et al., Blood 92:877-887, 1998), hybrid vectors (see, e.g., Choi, et al, Stem Cells 19:236-246, 2001), and complex retrovirus-derived vectors, such as lentiviral vectors.

In some embodiments, the B cells are contacted with a retroviral vector comprising a nucleic acid of interest operably linked to a promoter, under conditions sufficient to transduce at least a portion of the B cells. In one embodiment the B cells are contacted with a retroviral vector comprising a nucleic acid of interest operably linked to a promoter, under conditions sufficient to transduce at least 2% of the B cells. In some embodiments, the B cells are contacted with a vector under conditions sufficient to transduce at least 2%, 3%, 4%, 5%, 10%, 15%, 20%, 25%, 30%, 35%, 40%, 45%, 50%, 55%, 60%, 70%, 75%, 80%, 85%, 90%, 95%, 96%, 97%, 98%, 99% or even 100% of the resting B cells. In some embodiments, the differentiated and activated B cells, cultured in vitro as described herein, are transduced, in which case the cultured differentiated/activated B cells are contacted with a vector as described herein under conditions sufficient to transduce at least 2%, 3%, 4%, 5%, 10% 15%, 20%, 25%, 30%, 35%, 40%, 45%, 50%, 55%, 60%, 70%, 75%, 80%, 85%, 90%, 95%, 96%, 97%, 98%, 99% or even 100% of the differentiated and activated B cells.

In some embodiments, prior to transduction, the cells are prestimulated with Staphylococcus Aureus Cowan (SAC; Calbiochem, San Diego, CA) and/or IL-2 at appropriate concentrations known to the skilled person and routinely optimized. Other B cell activating factors (e.g., PMA), as are known to the skilled artisan and described herein may be used.

As noted above, some embodiments employ lentiviral vectors. The term “lentivirus” refers to a genus of complex retroviruses that are capable of infecting both dividing and non-dividing cells. Examples of lentiviruses include HIV (human immunodeficiency virus; including HIV type 1, and HIV type 2), visna-maedi, the caprine arthritis-encephalitis virus, equine infectious anemia virus, feline immunodeficiency virus (FIV), bovine immune deficiency virus (BIV), and simian immunodeficiency virus (SIV). Lentiviral vectors can be derived from any one or more of these lentiviruses (see, e.g., Evans et al, Hum Gene Ther. 10: 1479-1489, 1999; Case et al, PNAS USA 96:2988-2993, 1999; Uchida et al, PNAS USA 95: 1 1939-1 1944, 1998; Miyoshi et al, Science 283:682-686, 1999; Sutton et al, J Virol 72:5781-5788, 1998; and Frecha et al, Blood. 1 12:4843-52, 2008, each of which is incorporated by reference in its entirety).

It has been documented that resting T and B cells can be transduced by a VSVG-coated LV carrying most of the HIV accessory proteins (vif, vpr, vpu, and nee (see e.g., Frecha et al, 2010 Mol. Therapy 18: 1748). In certain embodiments the retroviral vector comprises certain minimal sequences from a lentivirus genome, such as the HIV genome or the SIV genome. The genome of a lentivirus is typically organized into a 5′ long terminal repeat (LTR) region, the gag gene, the pol gene, the env gene, the accessory genes (e.g., nef, vif, vpr, vpu, tat, rev) and a 3′ LTR region. The viral LTR is divided into three regions referred to as U3, R (repeat) and U5. The U3 region contains the enhancer and promoter elements, the U5 region contains the polyadenylation signals, and the R region separates the U3 and U5 regions. The transcribed sequences of the R region appear at both the 5′ and 3′ ends of the viral RNA (see, e.g., “RNA Viruses: A Practical Approach” (Alan J. Cann, Ed., Oxford University Press, 2000); O Narayan, J. Gen. Virology. 70: 1617-1639, 1989; Fields et al, Fundamental Virology Raven Press., 1990; Miyoshi et al, J Virol. 72:8150-7,1998; and U.S. Pat. No. 6,013,516, each of which is incorporated by reference in its entirety). Lentiviral vectors may comprise any one or more of these elements of the lentiviral genome, to regulate the activity of the vector as desired, or, they may contain deletions, insertions, substitutions, or mutations in one or more of these elements, such as to reduce the pathological effects of lentiviral replication, or to limit the lentiviral vector to a single round of infection.

Typically, a minimal retroviral vector comprises certain 5′LTR and 3′LTR sequences, one or more genes of interest (to be expressed in the target cell), one or more promoters, and a cis-acting sequence for packaging of the RNA. Other regulatory sequences can be included, as described herein and known in the art. The viral vector is typically cloned into a plasmid that may be transfected into a packaging cell line, such as a eukaryotic cell (e.g., 293-HEK), and also typically comprises sequences useful for replication of the plasmid in bacteria.

In certain embodiments, the viral vector comprises sequences from the 5′ and/or the 3′ LTRs of a retrovirus such as a lentivirus. The LTR sequences may be LTR sequences from any lentivirus from any species. For example, they may be LTR sequences from HIV, SIV, FIV or BIV. Preferably the LTR sequences are HIV LTR sequences. In certain embodiments, the viral vector comprises the R and U5 sequences from the 5′ LTR of a lentivirus and an inactivated or “self-inactivating” 3′ LTR from a lentivirus. A “self-inactivating 3′ LTR” is a 3′ long terminal repeat (LTR) that contains a mutation, substitution or deletion that prevents the LTR sequences from driving expression of a downstream gene. A copy of the U3 region from the 3′ LTR acts as a template for the generation of both LTR' s in the integrated provirus. Thus, when the 3′ LTR with an inactivating deletion or mutation integrates as the 5′ LTR of the provirus, no transcription from the 5′ LTR is possible. This eliminates competition between the viral enhancer/promoter and any internal enhancer/promoter. Self-inactivating 3′ LTRs are described, for example, in Zufferey et al, J Virol. 72:9873-9880, 1998; Miyoshi et al, J Virol. 72:8150-8157, 1998; and Iwakuma et al., J Virol. 261: 120-132, 1999, each of which is incorporated by reference in its entirety. Self-inactivating 3′ LTRs may be generated by any method known in the art. In certain embodiments, the U3 element of the 3′ LTR contains a deletion of its enhancer sequence, preferably the TATA box, Spl and/or NF-kappa B sites. As a result of the self-inactivating 3′ LTR, the provirus that is integrated into the host cell genome will comprise an inactivated 5′ LTR.

The vectors provided herein typically comprise a gene that encodes a protein (or other molecule, such as siRNA) that is desirably expressed in one or more target cells. In a viral vector, the gene of interest is preferably located between the 5′ LTR and 3′ LTR sequences. Further, the gene of interest is preferably in a functional relationship with other genetic elements, for example, transcription regulatory sequences such as promoters and/or enhancers, to regulate expression of the gene of interest in a particular manner once the gene is incorporated into the target cell. In certain embodiments, the useful transcriptional regulatory sequences are those that are highly regulated with respect to activity, both temporally and spatially.

In some embodiments, one or more additional genes may be incorporated as a safety measure, mainly to allow for the selective killing of transfected target cells within a heterogeneous population, such as within a human subject. In some embodiments, the selected gene is a thymidine kinase gene (TK), the expression of which renders a target cell susceptible to the action of the drug gancyclovir. In some embodiments, the suicide gene is a caspase 9 suicide gene activated by a dimerizing drug (see, e.g., Tey et al, Biology of Blood and Marrow Transplantation 13:913-924, 2007). In certain embodiments, a gene encoding a marker protein may be placed before or after the primary gene in a viral or non-viral vector to allow for identification and/or selection of cells that are expressing the desired protein. Certain embodiments incorporate a fluorescent marker protein, such as green fluorescent protein (GFP) or red fluorescent protein (RFP), along with the primary gene of interest. If one or more additional reporter genes are included, IRES sequences or 2A elements may also be included, separating the primary gene of interest from a reporter gene and/or any other gene of interest.

Certain embodiments may employ genes that encode one or more selectable markers. Examples include selectable markers that are effective in a eukaryotic cell or a prokaryotic cell, such as a gene for a drug resistance that encodes a factor necessary for the survival or growth of transformed host cells grown in a selective culture medium. Exemplary selection genes encode proteins that confer resistance to antibiotics or other toxins, e.g., G418, hygromycin B, puromycin, zeocin, ouabain, blasticidin, ampicillin, neomycin, methotrexate, or tetracycline, complement auxotrophic deficiencies, or supply may be present on a separate plasmid and introduced by co-transfection with the viral vector. In one embodiment, the gene encodes for a mutant dihydrofolate reductase (DHFR) that confers methotrexate resistance. Certain other embodiments may employ genes that encode one or cell surface receptors that can be used for tagging and detection or purification of transfected cells (e.g., low-affinity nerve growth factor receptor (LNGFR) or other such receptors useful as transduction tag systems. See e.g., Lauer et al., Cancer Gene Ther. 2000 March; 7(3):430-7.

Certain viral vectors such as retroviral vectors employ one or more heterologous promoters, enhancers, or both. In some embodiments, the U3 sequence from a retroviral or lentiviral 5′ LTR may be replaced with a promoter or enhancer sequence in the viral construct. Certain embodiments employ an “internal” promoter/enhancer that is located between the 5′ LTR and 3′ LTR sequences of the viral vector, and is operably linked to the gene of interest.

A “functional relationship” and “operably linked” mean, without limitation, that the gene is in the correct location and orientation with respect to the promoter and/or enhancer, such that expression of the gene will be affected when the promoter and/or enhancer is contacted with the appropriate regulatory molecules. Any enhancer/promoter combination may be used that either regulates (e.g., increases, decreases) expression of the viral RNA genome in the packaging cell line, regulates expression of the selected gene of interest in an infected target cell, or both.

A promoter is an expression control element formed by a DNA sequence that permits polymerase binding and transcription to occur. Promoters are untranslated sequences that are located upstream (5′) of the start codon of a selected gene of interest (typically within about 100 to 1000 bp) and control the transcription and translation of the coding polynucleotide sequence to which they are operably linked. Promoters may be inducible or constitutive. Inducible promoters initiate increased levels of transcription from DNA under their control in response to some change in culture conditions, such as a change in temperature. Promoters may be unidirectional or bidirectional. Bidirectional promoters can be used to co-express two genes, e.g., a gene of interest and a selection marker. Alternatively, a bidirectional promoter configuration comprising two promoters, each controlling expression of a different gene, in opposite orientation in the same vector may be utilized.

A variety of promoters are known in the art, as are methods for operably linking the promoter to the polynucleotide coding sequence. Both native promoter sequences and many heterologous promoters may be used to direct expression of the selected gene of interest. Certain embodiments employ heterologous promoters, because they generally permit greater transcription and higher yields of the desired protein as compared to the native promoter.

Certain embodiments may employ heterologous viral promoters. Examples of such promoters include those obtained from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). Certain embodiments may employ heterologous mammalian promoter, such as the actin promoter, an immunoglobulin promoter, a heat-shock promoter, or a promoter that is associated with the native sequence of the gene of interest. Typically, the promoter is compatible with the target cell, such as an activated B-lymphocyte, a plasma B cell, a memory B cell or other lymphocyte target cell.

Certain embodiments may employ one or more of the RNA polymerase II and III promoters. A suitable selection of RNA polymerase III promoters can be found, for example, in Paule and White. Nucleic Acids Research., Vol. 28, pp 1283-1298, 2000, which is incorporated by reference in its entirety. RNA polymerase II and III promoters also include any synthetic or engineered DNA fragments that can direct RNA polymerase II or III, respectively, to transcribe its downstream RNA coding sequences. Further, the RNA polymerase II or III (Pol II or III) promoter or promoters used as part of the viral vector can be inducible. Any suitable inducible Pol II or III promoter can be used with the methods described herein. Exemplary Pol II or III promoters include the tetracycline responsive promoters provided in Ohkawa and Taira, Human Gene Therapy, Vol. 11, pp 577-585, 2000; and Meissner et al, Nucleic Acids Research, Vol. 29, pp 1672-1682, 2001, each of which is incorporated by reference in its entirety.

Non-limiting examples of constitutive promoters that may be used include the promoter for ubiquitin, the CMV promoter (see, e.g., Karasuyama et al, J. Exp. Med. 169: 13, 1989), the β-actin (see, e.g., Gunning et al., PNAS USA 84:4831-4835, 1987), the elongation factor-1 alpha (EF-1 alpha) promoter, the CAG promoter, and the pgk promoter (see, e.g., Adra et al, Gene 60:65-74, 1987); Singer-Sam et al, Gene 32:409-417, 1984; and Dobson et al, Nucleic Acids Res. 10:2635-2637, 1982, each of which is incorporated by reference). Non-limiting examples of tissue specific promoters include the lck promoter (see, e.g., Garvin et al, Mol. Cell Biol. 8:3058-3064, 1988; and Takadera et al, Mol. Cell Biol. 9:2173-2180, 1989), the myogenin promoter (Yee et al, Genes and Development 7: 1277-1289. 1993), and the thyl promoter (see, e.g., Gundersen et al., Gene 1 13:207-214, 1992).

Additional examples of promoters include the ubiquitin-C promoter, the human μ heavy chain promoter or the Ig heavy chain promoter (e.g., MH), and the human K light chain promoter or the Ig light chain promoter (e.g., EEK), which are functional in B-lymphocytes. The MH promoter contains the human μ heavy chain promoter preceded by the \acute{ι}Eμ enhancer flanked by matrix association regions, and the EEK promoter contains the κ light chain promoter preceded an intronic enhancer (\acute{ι}Eκ), a matrix associated region, and a 3′ enhancer (3Eκ) (see, e.g., Luo et al, Blood. 1 13: 1422-1431, 2009, and U.S. Patent Application Publication No. 2010/0203630). Accordingly, certain embodiments may employ one or more of these promoter or enhancer elements.

In some embodiments, one promoter drives expression of a selectable marker and a second promoter drives expression of the gene of interest. For example, in one embodiment, the EF-1 alpha promoter drives the production of a selection marker (e.g., DHFR) and a miniature CAG promoter (see, e.g., Fan et al. Human Gene Therapy 10:2273-2285, 1999) drives expression of the gene of interest (e.g., IDUA). As noted above, certain embodiments employ enhancer elements, such as an internal enhancer, to increase expression of the gene of interest Enhancers are cis-acting elements of DNA, usually about 10 to 300 bp in length, that act on a promoter to increase its transcription Enhancer sequences may be derived from mammalian genes (e.g., globin, elastase, albumin, a-fetoprotein, insulin), such as the enhancer, the intronic enhancer, and the 3′ enhancer. Also included are enhancers from a eukaryotic virus, including the SV40 enhancer on the late side of the replication origin (bp 100-270), the cytomegalovirus early promoter enhancer, the polyoma enhancer on the late side of the replication origin, and adenovirus enhancers. Enhancers may be spliced into the vector at a position 5′ or 3′ to the antigen-specific polynucleotide sequence, but are preferably located at a site 5′ from the promoter. Persons of skill in the art will select the appropriate enhancer based on the desired expression partem.

In some embodiments, promoters are selected to allow for inducible expression of the gene. A number of systems for inducible expression are known in the art, including the tetracycline responsive system and the lac operator-repressor system. It is also contemplated that a combination of promoters may be used to obtain the desired expression of the gene of interest. The skilled artisan will be able to select a promoter based on the desired expression pattern of the gene in the organism and/or the target cell of interest.

Certain viral vectors contain cis-acting packaging sequences to promote incorporation of the genomic viral RNA into the viral particle. Examples include psi-sequences. Such cis-acting sequences are known in the art. In certain embodiments, the viral vectors described herein may express two or more genes, which may be accomplished, for example, by incorporating an internal promoter that is operably linked to each separate gene beyond the first gene, by incorporating an element that facilitates co-expression such as an internal ribosomal entry sequence (IRES) element (U.S. Pat. No. 4,937,190, incorporated by reference) or a 2A element, or both. Merely by way of illustration, IRES or 2A elements may be used when a single vector comprises sequences encoding each chain of an immunoglobulin molecule with a desired specificity. For instance, the first coding region (encoding either the heavy or light chain) may be located immediately downstream from the promoter, and the second coding region (encoding the other chain) may be located downstream from the first coding region, with an IRES or 2A element located between the first and second coding regions, preferably immediately preceding the second coding region. In some embodiments, an IRES or 2A element is used to co-express an unrelated gene, such as a reporter gene, a selectable marker, or a gene that enhances immune function. Examples of IRES sequences that can be used include, without limitation, the IRES elements of encephalomyelitis virus (EMCV), foot-and-mouth disease virus (FMDV), Theiler' s murine encephalomyelitis virus (TMEV), human rhinovirus (HRV), coxsackievirus (CSV), poliovirus (POLIO), Hepatitis A virus (HAV), Hepatitis C virus (HCV), and Pestiviruses (e.g., hog cholera virus (HOCV) and bovine viral diarrhea virus (BVDV)) (see, e.g., Le et al, Virus Genes 12: 135-147, 1996; and Le et al, Nuc. Acids Res. 25:362-369, 1997, each of which is incorporated by reference in their entirety). One example of a 2A element includes the F2A sequence from foot-and-mouth disease virus.

In some embodiments, the vectors provided herein also contain additional genetic elements to achieve a desired result. For example, certain viral vectors may include a signal that facilitates nuclear entry of the viral genome in the target cell, such as an HIV-1 flap signal. As a further example, certain viral vectors may include elements that facilitate the characterization of the provirus integration site in the target cell, such as a tRNA amber suppressor sequence. Certain viral vectors may contain one or more genetic elements designed to enhance expression of the gene of interest. For example, a woodchuck hepatitis virus responsive element (WRE) may be placed into the construct (see, e.g., Zufferey et al, J. Virol. 74:3668-3681, 1999; and Deglon et al, Hum. Gene Ther. 11 : 179-190, 2000, each of which is incorporated by reference in its entirety). As another example, a chicken β-globin insulator may also be included in the construct. This element has been shown to reduce the chance of silencing the integrated DNA in the target cell due to methylation and heterochromatinization effects. In addition, the insulator may shield the internal enhancer, promoter and exogenous gene from positive or negative positional effects from surrounding DNA at the integration site on the chromosome. Certain embodiments employ each of these genetic elements. In another embodiment, the viral vectors provided herein may also contain a Ubiquitous Chromatin Opening Element (UCOE) to increase expression (see e.g., Zhang F, et al, Molecular Therapy: The journal of the American Society of Gene Therapy 2010 September; 18(9): 1640-9.).

In some embodiments, the viral vectors (e.g., retroviral, lentiviral) provided herein are “pseudo-typed” with one or more selected viral glycoproteins or envelope proteins, mainly to target selected cell types. Pseudo-typing refers to generally to the incorporation of one or more heterologous viral glycoproteins onto the cell-surface virus particle, often allowing the virus particle to infect a selected cell that differs from its normal target cells. A “heterologous” element is derived from a virus other than the virus from which the RNA genome of the viral vector is derived. Typically, the glycoprotein-coding regions of the viral vector have been genetically altered such as by deletion to prevent expression of its own glycoprotein. Merely by way of illustration, the envelope glycoproteins gp41 and/or gpl20 from an HIV-derived lentiviral vector are typically deleted prior to pseudo-typing with a heterologous viral glycoprotein.

In some embodiments, the viral vector is pseudo-typed with a heterologous viral glycoprotein that targets B lymphocytes. In some embodiments, the viral glycoprotein allows selective infection or transduction of resting or quiescent B lymphocytes. In some embodiments, the viral glycoprotein allows selective infection of B lymphocyte plasma cells, plasmablasts, and activated B cells. In some embodiments, the viral glycoprotein allows infection or transduction of quiescent B lymphocytes, plasmablasts, plasma cells, and activated B cells. In some embodiments, viral glycoprotein allows infection of B cell chronic lymphocyte leukemia cells. In one embodiment, the viral vector is pseudo-typed with VSV-G. In some embodiments, the heterologous viral glycoprotein is derived from the glycoprotein of the measles virus, such as the Edmonton measles virus. In some embodiments, pseudo-type the measles virus glycoproteins hemagglutinin (H), fusion protein (F), or both (see, e.g., Frecha et al, Blood. 1 12:4843-52, 2008; and Frecha et al, Blood. 1 14:3173-80, 2009, each of which is incorporated by reference in its entirety). In some embodiments, the viral vector is pseudo-typed with gibbon ape leukemia virus (GALV). In some embodiments, the viral vector is pseudo-typed with cat endogenous retrovirus (RD114). In some embodiments, the viral vector is pseudo-typed with baboon endogenous retrovirus (BaEV). In some embodiments, the viral vector is pseudo-typed with murine leukemia virus (MLV). In some embodiments, the viral vector comprises an embedded antibody binding domain, such as one or more variable regions (e.g., heavy and light chain variable regions) which serves to target the vector to a particular cell type.

Generation of viral vectors can be accomplished using any suitable genetic engineering techniques known in the art, including, without limitation, the standard techniques of restriction endonuclease digestion, ligation, transformation, plasmid purification, PCR amplification, and DNA sequencing, for example as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)).

Any variety of methods known in the art may be used to produce suitable retroviral particles whose genome comprises an RNA copy of the viral vector. As one method, the viral vector may be introduced into a packaging cell line that packages the viral genomic RNA based on the viral vector into viral particles with a desired target cell specificity. The packaging cell line typically provides in trans the viral proteins that are required for packaging the viral genomic RNA into viral particles and infecting the target cell, including the structural gag proteins, the enzymatic pol proteins, and the envelope glycoproteins.

In some embodiments, the packaging cell line stably expresses certain necessary or desired viral proteins (e.g., gag, pol) (see, e.g., U.S. Pat. No. 6,218,181, herein incorporated by reference). In some embodiments, the packaging cell line is transiently transfected with plasmids that encode certain of the necessary or desired viral proteins (e.g., gag, pol, glycoprotein), including the measles virus glycoprotein sequences described herein. In some embodiments, the packaging cell line stably expresses the gag and pol sequences, and the cell line is then transfected with a plasmid encoding the viral vector and a plasmid encoding the glycoprotein. Following introduction of the desired plasmids, viral particles are collected and processed accordingly, such as by ultracentrifugation to achieve a concentrated stock of viral particles. Exemplary packaging cell lines include 293 (ATCC CCL X), HeLa (ATCC CCL 2), D17 (ATCC CCL 183), MDCK (ATCC CCL 34), BHK (ATCC CCL-10) and Cf2Th (ATCC CRL 1430) cell lines.

In some embodiments, the genetically modified B cell comprise a polynucleotide having a sequence that is identical to SEQ ID NO: 1. In some embodiments, the genetically modified B cells comprise a polynucleotide having a sequence that is at least about 85% identical to SEQ ID NO: 1, or at least about 90%, 95%, 96%, 97%, 98%, 99%, or greater than 99% identical to SEQ ID NO: 1.

In some embodiments, the genetically modified B cells are engineered on Day 2 or Day 3 after the start of culturing. In some embodiments, the genetically modified B cells are engineered using a method comprising electroporation. In some embodiments, the genetically modified B cells are harvested for administration to a subject on Day 4, Day 5, Day 6, or Day 7 in culture after engineering. In some embodiments, the genetically modified B cells are harvested for administration to a subject on Day 8 or later in culture after engineering. In some embodiments, the genetically modified B cells are harvested for administration to a subject on Day 10 or earlier in culture after engineering.

In some embodiments, the harvested genetically modified B cells do not produce significant levels of inflammatory cytokines. In some embodiments, the genetically modified B cells are harvested at a time-point in culture at which it is determined that they do not produce significant levels of inflammatory cytokines.

In some embodiments, the B cells are contacted with one or more B cell activating factors, e.g., any of a variety of cytokines, growth factors or cell lines known to activate and/or differentiate B cells (see e.g., Fluckiger, et al. Blood 1998 92: 4509-4520; Luo, et al, Blood 2009 1 13: 1422-1431). Such factors may be selected from the group consisting of, but not limited to, IL-1, IL-2, IL-3, IL-4, IL-5, IL-6, IL-7, IL-8, IL-9, IL-10, IL-1 1, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35, IFN-γ, IFN-a, IFN-β, IFN-δ, C type chemokines XCL1 and XCL2, C-C type chemokines (to date including CCL1 -CCL28) and CXC type chemokines (to date including CXCL1 -CXCL17), and members of the TNF superfamily (e.g., TNF-a, 4-1 BB ligand, B cell activating factor (BLyS), FAS ligand, sCD40L (including multimeric versions of sCD40L; e.g., histidine-tagged soluble recombinant CD40L in combination with anti-poly-histidine mAb to group multiple sCD40L molecules together), Lymphotoxin, OX40L, RANKL, TRAIL), CpG, and other toll like receptor agonists.

B cell activating factors may be added to in vitro cell cultures at various concentrations to achieve the desired outcome (e.g., expansion or differentiation). In some embodiment, a B cell activating factor is utilized in expanding the B cells in culture. In some embodiment, a B cell activating factor is utilized in differentiating the B cells in culture. In some embodiments, the B cell activating factor is utilized in both expanding and differentiating the B cells in culture. In some embodiments, the B cell activating factor is provided at the same concentration for expanding and differentiating. In some embodiments, the B cell activating factor is provided at a first concentration for expanding and at a second concentration for differentiating. It is contemplated that a B cell activating factor may be 1) utilized in expanding the B cells and not in differentiating the B cells, 2) utilized in differentiating the B cells and not in expanding the B cells, or 3) utilized in expanding and differentiating the B cells.

For example, B cells are cultured with one or more B cell activating factors selected from CD40L, IL-2, IL-4, and IL-10 for expansion of the B cells. In some embodiments, the B cells are cultured with 0.25-5.0 μg/ml CD40L. In some embodiments, the concentration of CD40L is 0.5 μg/ml. In one embodiment a crosslinking agent (such as an anti-HIS antibody in combination with HIS-tagged CD40L) is used to create multimers of CD40L. In some embodiments, molecules of CD40L are covalently linked or are held together using protein multimerization domains (e.g., the Fc region of an IgG or a leucine zipper domain). In some embodiments CD40L is conjugated to beads. In one embodiment CD40L is expressed from feeder cells. In some embodiments, the B cells are cultured with 1-10 ng/ml IL-2. In some embodiments, the concentration of IL-2 is 5 ng/ml. In one embodiment, the B cells are cultured with 1-10 ng/ml IL-4. In some embodiments, the concentration of IL-4 is 2 ng/ml. In some embodiments, the B cells are cultured with 10-100 ng/ml IL-10. In some embodiments, the concentration of IL-10 is 40 ng/ml.

In some embodiments, B cells are cultured with one or more B cell activating factors selected from CD40L, IL-2, IL-4, IL-10, IL-15 and IL-21 for expansion of the B cells. In some embodiments, the B cells are cultured with 0.25-5.0 μg/ml CD40L. In some embodiments, the concentration of CD40L is 0.5 μg/ml. In some embodiments a crosslinking agent (such as an anti-HIS antibody in combination with HIS-tagged CD40L) is used to create multimers of CD40L. In some embodiments, molecules of CD40L are covalently linked or are held together using protein multimerization domains (e.g., the Fc region of an IgG or a leucine zipper domain). In some embodiments, CD40L is conjugated to beads. In one embodiment CD40L is expressed from feeder cells. In one embodiment, the B cells are cultured with 1-10 ng/ml IL-2. In some embodiments, the concentration of IL-2 is 5 ng/ml. In some embodiments, the B cells are cultured with 1-10 ng/ml IL-4. In some embodiments, the concentration of IL-4 is 2 ng/ml. In one embodiment, the B cells are cultured with 10-100 ng/ml IL-10. In some embodiments, the concentration of IL-10 is 40 ng/ml. In one embodiment, the B cells are cultured with 50-150 ng/ml IL-15. In some embodiments, the concentration of IL-15 is 100 ng/ml. In some embodiments, the B cells are cultured with 50-150 ng/ml IL-21. In some embodiments, the concentration of IL-21 is 100 ng/ml. In some embodiments, the B cells are cultured with CD40L, IL-2, IL-4, IL-10, IL-15 and IL-21 for expansion of the B cells.

In some embodiments, the genetically modified B cells are grown in a culture system that comprises each of IL-2, IL-4, IL-10, IL-15, IL-31, and a multimerized CD40 ligand throughout the entire culture period pre- and post-engineering. In some embodiments, the multimerized CD40 ligand is a HIS tagged CD40 ligand that is multimerized using an anti-his antibody.

For example, in one embodiment, B cells are cultured with the B cell activating factors CD40L, IL-2, IL-4, IL-10, IL-15 and IL-21 for expansion of the B cells, wherein the CD40L is crosslinked with a crosslinking agent to create multimers of CD40L. Such a culture system may be maintained throughout an entire culture period (e.g., a 7 day culture period), in which the B cells are transfected or otherwise engineered to express a transgene of interest (e.g., an exogenous polypeptide such as, e.g., IDUA).

In another example, B cells are cultured with one or more B cell activating factors selected from CD40L, IFN-a, IL-2, IL-6, IL-10, IL-15, IL-21, and P-class CpG oligodeoxynucleotides (p-ODN) for differentiation of the B cells. In some embodiments, the B cells are cultured with 25-75 ng/ml CD40L. In some embodiments, the concentration of CD40L is 50 ng/ml. In some embodiments, the B cells are cultured with 250-750 U/ml IFN-a. In some embodiments, the concentration of the IFN-a is 500 U/ml. In some embodiments, the B cells are cultured with 5-50 U/ml IL-2. In some embodiments, the concentration of IL-2 is 20 U/ml. In some embodiments, the B cells are cultured with 25-75 ng/ml IL-6. In some embodiments, the concentration of IL-6 is 50 ng/ml. In one embodiment, the B cells are cultured with 10-100 ng/ml IL-10. In some embodiments, the concentration of IL-10 is 50 ng/ml. In some embodiments, the B cells are cultured with 1-20 ng/ml IL-15. In one embodiment, the concentration of IL-15 is 10 ng/ml. In some embodiments, the B cells are cultured with 10-100 ng/ml IL-21. In some embodiments, the concentration of IL-21 is 50 ng/ml. In one embodiment, the B cells are cultured with 1-50 μg/ml p-ODN. In some embodiments, the concentration of p-ODN is 10 μg/ml.

In some embodiments, B cells are contacted or cultured on feeder cells. In some embodiments, the feeder cells are a stromal cell line, e.g., murine stromal cell line S 17 or MS5. In some embodiments, isolated CD19+ cells are cultured with one or more B cell activating factor cytokines, such as IL-10 and IL-4, in the presence of fibroblasts expressing CD40-ligand (CD40L, CD154). In some embodiments, CD40L is provided bound to a surface such as tissue culture plate or a bead. In some embodiments, purified B cells are cultured, in the presence or absence of feeder cells, with CD40L and one or more cytokines or factors selected from IL-10, IL-4, IL-7, p-ODN, CpG DNA, IL-2, IL-15, IL6, IL-21, and IFN-a.

In some embodiments, B cell activating factors are provided by transfection into the B cell or other feeder cell. In this context, one or more factors that promote differentiation of the B cell into an antibody secreting cell and/or one or more factors that promote the longevity of the antibody producing cell may be used. Such factors include, for example, Blimp-1, TRF4, anti-apoptotic factors like Bcl-xl or Bcl5, or constitutively active mutants of the CD40 receptor. Further, factors which promote the expression of downstream signaling molecules such as TNF receptor-associated factors (TRAFs) may also be used in the activation/differentiation of the B cells. In this regard, cell activation, cell survival, and antiapoptotic functions of the TNF receptor superfamily are mostly mediated by TRAF1-6 (see e.g., R. H. Arch, et al, Genes Dev. 12 (1998), pp. 2821-2830). Downstream effectors of TRAF signaling include transcription factors in the NF-KB and AP-1 family which can turn on genes involved in various aspects of cellular and immune functions. Further, the activation of NF-κB and AP-1 has been shown to provide cells protection from apoptosis via the transcription of antiapoptotic genes.

In some embodiments, Epstein Barr virus (EBV)-derived proteins are used for the activation and/or differentiation of B cells or to promote the longevity of the antibody producing cell. EBV-derived proteins include but are not limited to, EBNA-1, EBNA-2, EBNA-3, LMP-1, LMP-2, EBER, miRNAs, EBV-EA, EBV-MA, EBV-VCA and EBV-AN.

In some embodiments, contacting the B cells with B cell activation factors using the methods provided herein leads to, among other things, cell proliferation (i.e., expansion), modulation of the IgM+ cell surface phenotype to one consistent with an activated mature B cell, secretion of Ig, and isotype switching. CD19+ B cells may be isolated using known and commercially available cell separation kits, such as the MiniMACS™ cell separation system (Miltenyi Biotech, Bergisch Gladbach, Germany) In certain embodiments, CD40L fibroblasts are irradiated before use in the methods described herein. In one embodiment, B cells are cultured in the presence of one or more of IL-3, IL-7, Flt3 ligand, thrombopoietin, SCF, IL-2, IL-10, G-CSF and CpG. In some embodiments, the methods include culturing the B cells in the presence of one or more of the aforementioned factors in conjunction with transformed stromal cells (e.g., MS5) providing a low level of anchored CD40L and/or CD40L bound to a plate or a bead.

As discussed above, B cell activating factors induce expansion, proliferation, or differentiation of B cells. Accordingly, B cells are contacted with one or more B cell activating factors listed above to obtain an expanded cell population. A cell population may be expanded prior to transfection. Alternatively, or additionally, a cell population may be expanded following transfection. In one embodiment, expanding a B cell population comprises culturing cells with IL-2, IL-4, IL-10 and CD40L (see e.g., Neron et al. PLoS ONE, 2012 7(12):e51946). In one embodiment, expanding a B cell population comprises culturing cells with IL-2, IL-10, CpG, and CD40L. In one embodiment, expanding a B cell population comprises culturing cells with IL-2, IL-4, IL-10, IL-15, IL-21, and CD40L. In one embodiment, expanding a B cell population comprises culturing cells with IL-2, IL-4, IL-10, IL-15, IL-21, and multimerized CD40L.

In some embodiments, expansion of a B cell population is induced and/or enhanced by a small molecule compound added to the cell culture. For example, a compound that binds to and dimerizes CD40 can be used to trigger the CD40 signaling pathway.

Any of a variety of culture media may be used in the present methods as would be known to the skilled person (see e.g., Current Protocols in Cell Culture, 2000-2009 by John Wiley & Sons, Inc.). In some embodiments, media for use in the methods described herein includes, but is not limited to Iscove modified Dulbecco medium (with or without fetal bovine or other appropriate serum). Illustrative media also includes, but is not limited to, IMDM, RPMI 1640, AIM-V, DMEM, MEM, a-MEM, F-12, X-Vivo 15, and X-Vivo 20. In some embodiments, the medium may comprise a surfactant, an antibody, plasmanate or a reducing agent (e.g. N-acetyl-cysteine, 2-mercaptoethanol), one or more antibiotics, and/or additives such as insulin, transferrin, sodium selenite and cyclosporin. In some embodiments, IL-6, soluble CD40L, and a cross-linking enhancer may also be used.

B cells are cultured under conditions and for sufficient time periods to achieve differentiation and/or activation desired. In some embodiments, the B cells are cultured under conditions and for sufficient time periods such that 10%, 15%, 20%, 25%, 30%, 35%, 40%, 45%, 50%, 55%, 60%, 65%, 70%, 75%, 80%, 85%, 90%, 95%, or even 100% of the B cells are differentiated and/or activated as desired. In some embodiments, the B cells are activated and differentiated into a mixed population of plasmablasts and plasma cells. As would be recognized by the skilled person, plasmablasts and plasma cells may be identified by cell surface protein expression patterns using standard flow cytometry methods as described elsewhere herein, such as expression of one or more of CD38, CD78, IL-6R, CD27^{high}, and CD138 and/or lack of, or reduction of, expression of one or more of CD19, CD20 and CD45. As would be understood by the skilled person, memory B cells are generally CD20+ CD19+ CD27+ CD38− while early plasmablasts are CD20− CD19+ CD27++ CD38++. In one embodiment, the cells cultured using the methods described herein are CD20−, CD38+, CD138−. In another embodiment, the cells have a phenotype of CD20−, CD38+, CD138+. In certain embodiments, cells are cultured for 1-7 days. In further embodiments, cells are cultured 7, 14, 21 days or longer. Thus, cells may be cultured under appropriate conditions for 1, 2, 3, 4, 5, 6, 7, 8, 9, 10, 11, 12, 13, 14, 15, 16, 17, 18, 19, 20, 21, 22, 23, 24, 25, 26, 27, 28, 29, or more days. Cells are re-plated, and media and supplements may be added or changed as needed using techniques known in the art.

In some embodiments, the B cells are cultured under conditions and for sufficient time periods such that at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the cells are differentiated and activated to produce Ig and/or to express the transgene.

The induction of B cell activation may be measured by techniques such as H-uridine incorporation into RNA (as B cells differentiate, RNA synthesis increases), or by H-thymidine incorporation, which measures DNA synthesis associated with cell proliferation. In some embodiments, interleukin-4 (IL-4) may be added to the culture medium at an appropriate concentration (e.g., about 10 ng/ml) for enhancement of B cell proliferation.

Alternatively, B cell activation is measured as a function of immunoglobulin secretion. For example, CD40L is added to resting B cells together with IL-4 (e.g., 10 ng/ml) and IL-5 (e.g., 5 ng/ml) or other cytokines that activate B cells. Flow cytometry may also be used for measuring cell surface markers typical of activated B cells. See e.g., Civin C I, Loken M R, Int'l J. Cell Cloning 987; 5: 1-16; Loken, M R, et al, Flow Cytometry Characterization of Erythroid, Lymphoid and Monomyeloid Lineages in Normal Human Bone Marrow, in Flow Cytometry in Hematology, Laerum O D, Bjerksnes R. eds., Academic Press, New York 1992; pp. 31 -42; and LeBein T W, et ai, Leukemia 1990; 4:354-358.

After culture for an appropriate period of time, such as from 2, 3, 4, 5, 6, 7, 8, 9, or more days, generally around 3 days, an additional volume of culture medium may be added. Supernatant from individual cultures may be harvested at various times during culture and quantitated for IgM and IgGl as described in Noelle et al, (1991) J. Immunol. 146: 1118-1124. In some embodiments, the culture is harvested and measured for expression of the transgene of interest using flow cytometry, enzyme-linked immunosorbent assay (ELISA), ELISPOT or other assay known in the art. In some embodiments, ELISA is used to measure antibody isotype production, e.g., IgM, or a product of the transgene of interest. In some embodiments, IgG determinations are made using commercially available antibodies, such as goat anti-human IgG, as capture antibody followed by detection using any of a variety of appropriate detection reagents such as biotinylated goat antihuman Ig, streptavidin alkaline phosphatase and substrate.

In certain embodiments, the B cells are cultured under conditions and for sufficient time periods such that the number of cells is 1, 10, 25, 50, 75, 100, 125, 150, 175, 200, 250, 300, 350, 400, 450, 500, 550, 600, 650, 700, 750, 800, 850, 900, 950, 1000 fold or more greater than the number of B cells at the start of culture. In some embodiments, the number of cells is 10 to 1000-fold greater, including consecutive integers therein, than the number of B cells at the start of culture. For example, an expanded B cell population is at least 10-fold greater than the initial isolated B cell population. In another embodiment, the expanded B cell population is at least 100-fold greater than the initial isolated B cell population. In some embodiments, the expanded B cell population is at least 500-fold greater than the initial isolated B cell population.

In some embodiments, the method comprises expanding the genetically modified B cells prior to the administering to the subject.

In some embodiments, an engineered B cell population is assessed for polyclonality prior to administration to a subject. Ensuring polyclonality of the final cell product is an important safety parameter. Specifically, the emergence of a dominant clone is viewed as potentially contributing to in vivo tumorigenesis or auto-immune disease. Polyclonality may be assessed by any means known in the art or described herein. For example, in some embodiments, polyclonality is assessed by sequencing (e.g., by deep sequencing) the B cell receptors expressed in an engineered B cell population. Since the B cell receptor undergoes changes during B cell development that makes it unique between B cells, this method allows for quantifying how many cells share the same B cell receptor sequence (meaning they are clonal). Thus, in some embodiments, the more B cells in an engineered B cell population that express the same B cell receptor sequence, the more clonal the population and, therefore, the less safe the population is for administration to a subject. Conversely, in some embodiments, the less B cells in an engineered B cell population that express the same B cell receptor sequence, the less clonal the population (i.e., more polyclonal) and, thus, the more safe the population is for administration to a subject.

In some embodiments, an engineered B cell population is assessed for polyclonality prior to administration to a subject. Ensuring polyclonality of the final cell product is an important safety parameter. Specifically, the emergence of a dominant clone is viewed as potentially contributing to in vivo tumorigenesis or auto-immune disease. Polyclonality may be assessed by any means known in the art or described herein. For example, in some embodiments, polyclonality is assessed by sequencing (e.g., by deep sequencing) the B cell receptors expressed in an engineered B cell population. Since the B cell receptor undergoes changes during B cell development that makes it unique between B cells, this method allows for quantifying how many cells share the same B cell receptor sequence (meaning they are clonal). Thus, in some embodiments, the more B cells in an engineered B cell population that express the same B cell receptor sequence, the more clonal the population and, therefore, the less safe the population is for administration to a subject. Conversely, in some embodiments, the less B cells in an engineered B cell population that express the same B cell receptor sequence, the less clonal the population (i.e., more polyclonal) and, thus, the more safe the population is for administration to a subject.

In some embodiments, the engineered B cells are administered to a subject after they have been determined to be sufficiently polyclonal. For example, the engineered B cells may be administered to a subject after it has been determined that no particular B cell clone in the final population comprises more than about 0.2% of the total B cell population. The engineered B cells may be administered to a subject after it has been determined that no particular B cell clone in the final population comprises more than about 0.1% of the total B cell population, or more than about 0.09%, 0.08%, 0.07%, 0.06%, 0.05%, or about 0.04%, of the total B cell population. In particular embodiments, the engineered B cells (e.g., which produce IDUA) are administered to a subject after it has been determined that no particular B cell clone in the final population comprises more than about 0.03% of the total B cell population.

In some embodiments, the final population of expanded genetically modified B cells demonstrates a high degree of polyclonality. In some embodiments, any particular B cell clone in the final population of expanded genetically modified B cells comprises less than 0.2% of the total B cell population. In some embodiments, any particular B cell clone in the final population of expanded genetically modified B cells comprises less than 0.05% of the total B cell population.

In some embodiments, the genetically modified B cells comprise a polynucleotide encoding a human DHFR gene with enhanced resistance to methotrexate. In some embodiments, the human DHFR gene with enhanced resistance to methotrexate contains a substitution of leucine to tyrosine at amino acid 22 and a substitution of phenylalanine to serine at amino acid 31. In some embodiments, the method comprises treating the genetically modified B cells with methotrexate prior to harvesting for administration. In some embodiments, the methotrexate treatment is between 100 nM and 300 nM. In some embodiments, the methotrexate treatment is 200 nM.

In some embodiments, the genetically modified B cells travel throughout tissues within the central nervous system (CNS) upon administration to the subject. In some embodiments, the administration of the genetically modified B cells to the subject results in the reduction of glycosaminoglycans (GAGs) in diverse tissues of the subject. In some embodiments, the administration of the genetically modified B cells to the subject results in the reduction of GAGs in tissues within the central nervous system (CNS).

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

The materials and methods employed in these experiments are now described.

### Example 1: Administration of Engineered B Cells to the Central Nervous System Via Direct Injection into Cerebrospinal Fluid

### Background

Mucopolysaccharidosis type I (MPSI) is a lysosomal storage disease caused by deficiency in the enzyme alpha-L-iduronidase (IDUA). IDUA catalyzes the breakdown of glycosaminoglycans (GAG) heparan sulfate and dermatan sulfate in the body. Lack of IDUA leads to accumulation of GAG in all tissues, including the brain.

Current therapies include hematopoietic stem cell transplant and enzyme replacement. Both restore systemic IDUA activity and reduce the level of accumulated GAG in the periphery. While these treatments significantly expand life expectancy and improve quality of life, a significant burden of disease remains. Particularly cognitive impairments prove to be resistant to treatment, necessitating the development of therapies that directly target the CNS.

NSG-MPSI mice are immune deficient and lack IDUA expression. The mice recapitulate many of the manifestations of human MPSI, including lack of IDUA activity and high GAG levels.

To specifically address the lack of IDUA in the CNS, human B cells genetically engineered to express IDUA were introduced into the lateral ventricles of the animals.

### Methods

NSG-MPS I mice (4 months of age, n=6) were injected ICV (one lateral ventricle) with 2e5 ISP-001 B cells. Mice were euthanized at day 1 (n=1), day 7 (n=1), day 28 (n=2) and day 42 (n=4) post injection. Non-treated NSG-MPS I mice (n=2) served as negative controls. Brain tissue was analyzed for IDUA activity and GAG levels. For the IDUA enzyme assay brain tissue was lysed and assayed for IDUA activity in a fluorometric assay using 4-methylumbelliferyl-alpha-L-iduronide as substrate (Glycosynth, England). Protein concentration of the tissue lysates were determined using the Pierce assay system. IDUA activity was recorded as nmol/h/mg protein. For the GAG assay, brain lysates were assayed using the Blyscan™ Sulfated Glycosaminoglycan Assay kit (Biocolor Life Science Assays; Accurate Chemical). Tissue GAG content was reported in micrograms GAG per milligram protein.

### Results

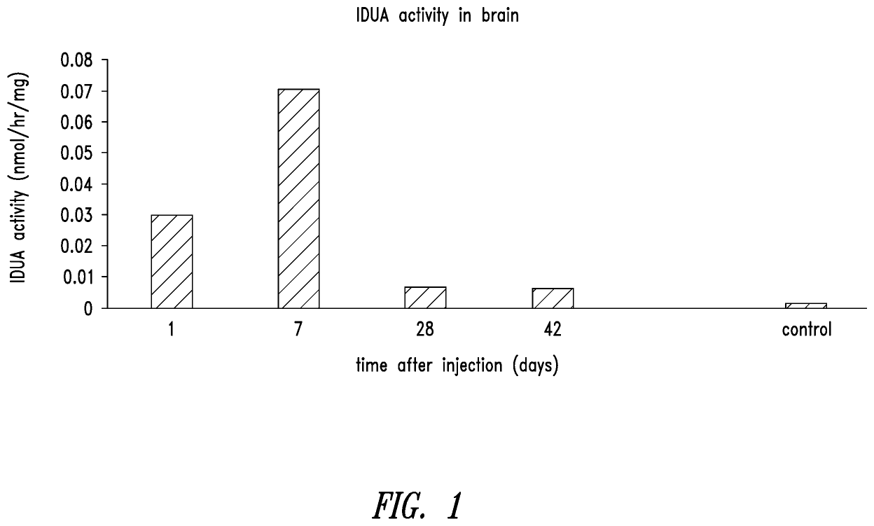

Extracts of whole brain were assayed for IDUA activity (FIG. 1) using fluorometric methodology. We observed significant IDUA activity in the treated animals as compared to the control animals. The enzyme activity was highest seven days post ICV injection, but was still detectable on days 28 and 42.

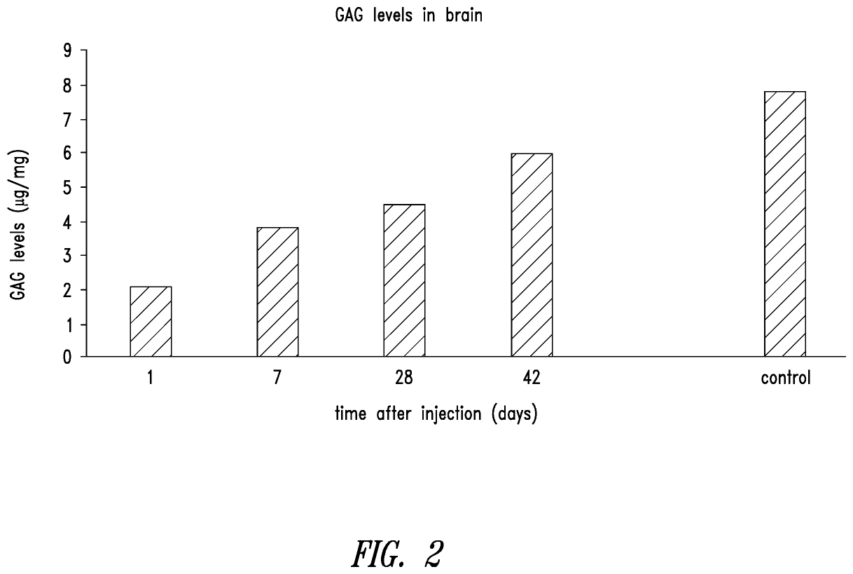

Extracts of whole brain were assayed for GAG levels (FIG. 2). GAG levels in brain tissue were lower in all treated animals as compared to control animals. The levels were lowest one day after injection, but remained below those observed in control animals for the duration of the study.

### Discussion

This study demonstrates delivering therapeutic B cells to the CNS by infusion into the cerebrospinal fluid (CSF), in this case by ICV injection. Secretion of IDUA peaked one week post injection, but IDUA levels remained detectable for the duration of the study (six weeks). The presence of IDUA was also reflected in the significant reduction of GAG levels in the brain.

### Example 2: Xeno-Adoptive Transfer of Modified Human B Cells into Mouse by Intracerebroventricular Injection

### Methods

Human B cells were transposed with LUC following standard protocols. NSG mice (n=4, female) were injected I.P. with 3e6 CD4+ T cells isolated from the same donor. One week after preconditioning with T cells, mice were injected in both lateral ventricles with LUC-transposed human B cells (4e5/ventricle). Engraftment of B cells was monitored biweekly by bioluminescence imaging (IVIS). The study ended at day 16 (after B cell injection).

### Results

Mice were imaged starting 2 days after B cell injection (Feb. 26, 2021) and biweekly thereafter. No adverse effects were noticed.

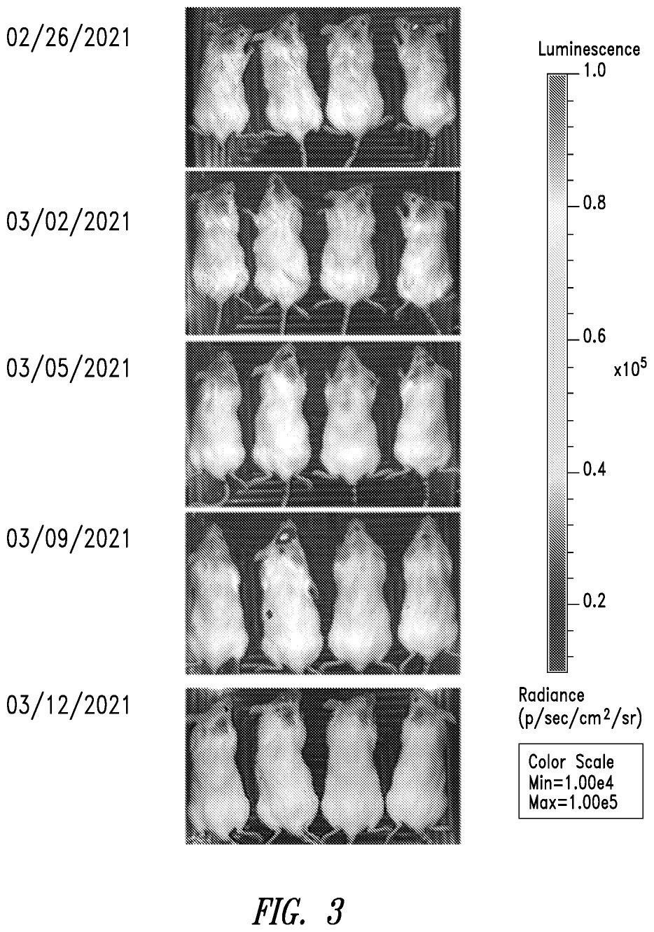

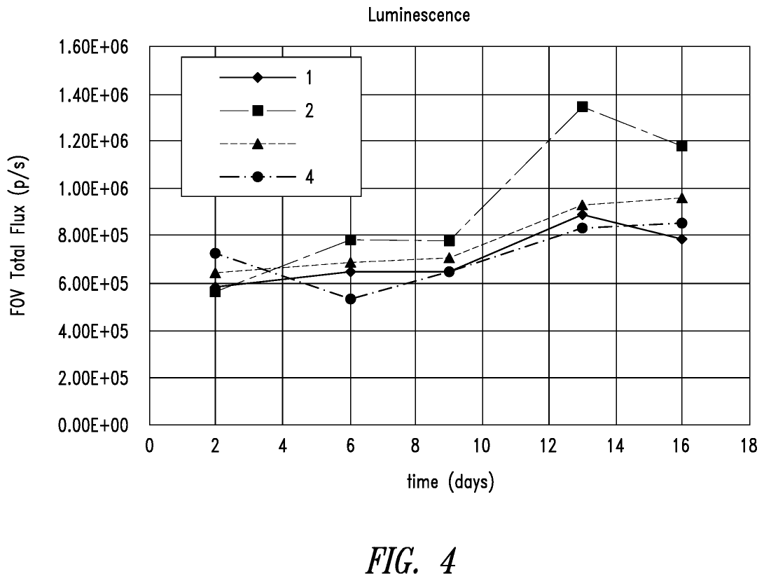

The results are shown in FIGS. 3 and 4. Luminescence signal was detected in all animals, however at different time points and with different intensities. Mouse 1 showed overall the lowest signal, with the highest signal 13 days after injection. Mouse 2 showed the highest signal in all animals, with the exception of the first imaging. The signal increased to the highest level on day 13, after which it decreased somewhat. Animal 3 showed a steady increase of luminescence throughout the study and mouse 4 showed steady increase of luminescence after an initial drop at day 6. The luminescence signal in all animals appeared to stay localized to the ventricles, although mouse 2 showed some signal in the spleen area on day 13.

### Discussion

Luminescence signal was detected in the area of the vesicles throughout the study, indicating successful engraftment of the B cells. The signal showed inter-animal variability, but appeared to increase over time, suggesting expansion of the B cells. This experiment evidences that human B cells can successful engraft in the lateral ventricles following intracerebroventricular (ICV) administration to the CNS.

What is claimed is:

1. A method of administering genetically modified B cells to a subject for in vivo production of a therapeutic agent comprising:
administering one or more doses of genetically modified B cells to a subject's central nervous system (CNS).

2. The method of claim 1, wherein the administering comprises infusion into the cerebrospinal fluid (CSF) of the subject.

3. The method of claim 2, wherein the administering comprises intracisternal injection.

4. The method of claim 2, wherein the administering comprises intrathecal injection.

5. The method of claim 2, wherein the administering comprises intracerebroventricular injection (ICV).

6. The method of claim 5, wherein the intracerebroventricular injection (ICV) occurs in one or more brain cavities.

7. The method of claim 6, wherein the one or more brain cavities is a lateral ventricle.

8. The method of claim 6, wherein the one or more brain cavities is a third ventricle.

9. The method of claim 6, wherein the one or more brain cavities is a cerebral aqueduct.

10. The method of claim 6, wherein the one or more brain cavities is a fourth ventricle.

11. The method of claim 1, wherein the therapeutic agent produced by the genetically modified B cells is iduronidase (IDUA).

12. The method of claim 1, wherein doses comprise the genetically modified B cells at sub-optimal single-dose concentrations, wherein sub-optimal single dose concentrations are determined by:
(i) testing multiple single doses of the modified B cells;
(ii) determining an optimal single-dose concentration of the modified B cells,
wherein increasing the dosage of modified B cells present in a single-dose concentration of modified B cells results in the production of the therapeutic agent;
(iii) testing multiple sub-optimal single dose concentrations of the modified B cells; and
(iv) determining a sub-optimal single-dose of the modified B cells,
wherein the resulting dosage results in a greater than linear increase over lower dosages,
wherein the sub-optimal single-dose concentration is less than or equivalent to about one half or about one third the dose of the optimal single-dose concentration.

13. The method of claim 1, wherein the administering optionally comprises one or more sequential doses of the genetically modified B cells.

14. The method of claim 1, wherein the subject is a mammal.

15. The method of claim 1, wherein the subject is a human.

16. The method of claim 1, wherein the genetically modified B cells are autologous to the subject.

17. The method of claim 1, wherein the genetically modified B cells are allogeneic to the subject.

18. The method of claim 1, wherein the therapeutic agent is a protein.

19. The method of claim 18, wherein the protein is an enzyme.

20. The method of claim 1, wherein the genetically modified B cells are CD20−, CD38+, and CD138+.

21. The method of claim 1, wherein the genetically modified B cells are CD20−, CD38+, and CD138−.

22. The method of claim 1, wherein the genetically modified B cells were prepared using a Sleeping Beauty transposon to express the therapeutic agent in the B cells.

23. The method of claim 1, wherein the genetically modified B cells were prepared using a recombinant viral vector to express the therapeutic agent in the B cells.

24. The method of claim 23, wherein the recombinant viral vector encodes a recombinant retrovirus, recombinant lentivirus, recombinant adenovirus, or recombinant adeno-associated virus.

25. The method of claim 1, wherein the genetically modified B cells were prepared by gene editing of the B cell genome or by targeted integration into the genome of the B cell a polynucleotide sequence encoding the therapeutic agent.

26. The method of claim 25, wherein the targeted integration comprises a zinc finger nuclease-mediated gene integration, CRISPR-mediated gene integration or gene editing, TALE-nuclease-mediated gene integration, or meganuclease-mediated gene integration.

27. The method of claim 26, wherein the targeted integration of polynucleotide occurred via homologous recombination.

28. The method of claim 25, wherein the targeted integration comprises a viral vector-mediated delivery of a nuclease capable of inducing a DNA cleavage at a target site.

29. The method of claim 28, wherein the nuclease is a zinc finger nuclease, a Cas nuclease, a TALE-nuclease, or a meganuclease.

30. The method of any one of claims 1-29, wherein the genetically modified B cell comprise a polynucleotide having a sequence that is identical to SEQ ID NO: 1.

31. The method of any one of claims 1-29, wherein the genetically modified B cells comprise a polynucleotide having a sequence that is at least about 85% identical to SEQ ID NO: 1, or at least about 90%, 95%, 96%, 97%, 98%, 99%, or greater than 99% identical to SEQ ID NO: 1.

32. The method of any one of claims 1-31, wherein the genetically modified B cells are engineered on Day 2 or Day 3 after the start of culturing.

33. The method of claim 32, wherein the genetically modified B cells are engineered using a method comprising electroporation.

34. The method of any one of claims 1-33, wherein the genetically modified B cells are harvested for administration to a subject on Day 4, Day 5, Day 6, or Day 7 in culture after engineering.

35. The method of any one of claims 1-33, wherein the genetically modified B cells are harvested for administration to a subject on Day 8 or later in culture after engineering.

36. The method of claim 35, wherein the genetically modified B cells are harvested for administration to a subject on Day 10 or earlier in culture after engineering.

37. The method of any one of claims 1-36, wherein the harvested genetically modified B cells do not produce significant levels of inflammatory cytokines.

38. The method of any one of claims 1-36, wherein the genetically modified B cells are harvested at a time-point in culture at which it is determined that they do not produce significant levels of inflammatory cytokines.

39. The method of any one of claims 1-38, wherein the genetically modified B cells are grown in a culture system that comprises each of IL-2, IL-4, IL-10, IL-15, IL-21, and a multimerized CD40 ligand throughout the entire culture period pre- and post-engineering.

40. The method of claim 39, wherein the multimerized CD40 ligand is a HIS tagged CD40 ligand that is multimerized using an anti-his antibody.

41. The method of any one of claims 1-40, further comprising expanding the genetically modified B cells prior to the administering to the subject.

42. The method of claim 41, wherein the final population of expanded genetically modified B cells demonstrates a high degree of polyclonality.

43. The method of claim 41, wherein any particular B cell clone in the final population of expanded genetically modified B cells comprises less than 0.2% of the total B cell population.

44. The method of claim 41, wherein any particular B cell clone in the final population of expanded genetically modified B cells comprises less than 0.05% of the total B cell population.

45. The method of any one of claims 1-44, wherein the genetically modified B cells comprise a polynucleotide encoding a human DHFR gene with enhanced resistance to methotrexate.

46. The method of claim 45, wherein the human DHFR gene with enhanced resistance to methotrexate contains a substitution of leucine to tyrosine at amino acid 22 and a substitution of phenylalanine to serine at amino acid 31.

47. The method of any one of claims 1-46, comprising treating the genetically modified B cells with methotrexate prior to harvesting for administration.

48. The method of claim 47, wherein the methotrexate treatment is between 100 nM and 300 nM.

49. The method of claim 48, wherein the methotrexate treatment is 200 nM.

50. The method of any one of claims 1-49, wherein the genetically modified B cells travel throughout tissues within the central nervous system (CNS) upon administration to the subject.

51. The method of claims 50, wherein the administration of the genetically modified B cells to the subject results in the reduction of glycosaminoglycans (GAGs) in diverse tissues of the subject.

52. The method of claims 51, wherein the administration of the genetically modified B cells to the subject results in the reduction of GAGs in tissues within the central nervous system (CNS).

53. The method of any one of claims 1-52, wherein the genetically modified B cells persist in the central nervous system (CNS) for at least about one week, two weeks, three weeks, four weeks, five weeks, or six weeks post administration.

54. The method of any one of claims 1-52, wherein the genetically modified B cells persist in the central nervous system (CNS) for at least about one month, two months, three months, four months, five months, or six months post administration.

55. The method of any one of claims 1-52, wherein the genetically modified B cells persist in the central nervous system (CNS) for at most about one week, two weeks, three weeks, four weeks, five weeks, or six weeks post administration.

56. The method of any one of claims 1-52, wherein the genetically modified B cells persist in the central nervous system (CNS) for at most about one month, two months, three months, four months, five months, or six months post administration.

57. The method of any one of claims 1-56, wherein the subject has a disease or disorder associated with lysosomal storage dysfunction.

58. The method of claim 57, wherein the disease or disorder associated with lysosomal storage dysfunction is caused by enzyme alpha-L-iduronidase (IDUA) deficiency.

59. The method of claim 57, wherein the subject has mucopolysaccharidosis type I (MPS I).

60. The method of any one of claims 57-59, wherein the administration of the genetically modified B cells treats the subject's disease or disorder associated with lysosomal storage dysfunction.

61. The method of claim 59, wherein the administration of the genetically modified B cells treats the subject's MPS I.


SEQUENCE LISTING (1 sequence)

SEQ ID NO: 1 - DNA, 7322 bp, Artificial Sequence
  Made in Lab - Plasmid construct pKT2/EEK-IDUA- DHFR
cctggatccagatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaagctgcgcactaggcaagttaactaactcctctgaatgtcagtatttccatctgtaagatgaacacagtggggctccaattccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggccagctagagcggccgcttaatcattcttctcatatacttcaaatttgtacttaatgcctttctcctcctggacatcagagagaacacctgggtattctggcagaagtttatatttctccaaatcaatttctggaaaaaacgtgtcactttcaaagtcttgcatgatccttgtcacaaatagtttaagatggcctgggtgattcatggcttccttataaacagaactgccaccaactatccagaccatgtctactttatttgctaattctggttgttcagtaagttttaaggcatcatctagacttctggaaagaaaatgagctccttgtggaggttccttgagttctctgctgagaactaaattaattctaccctttaaaggtcgattcttctcaggaatggagaaccaggtcttcttacccataatcaccagattctgtttaccttctactgaagaggttgtggtcattctctggaaatatctggattcattcctgagcggtggccagggatagtccccgttcttgccgatgcccatgttctgggacacagcgacgatgcagtttagcgaaccaaccatgatggaagctactgtacaccaacctgtcaggagaggaaagagaagaaggttagtacaattgtctagggctgcagggttcatagtgccacttttcctgcactgccccatctcctgcccaccctttcccaggcatagacagtcagtgacttaccaaactcacaggagggagaaggcagaagcttgaatgttcacagagactactgcacttatatatggttctcccccaccctggggaaaaaggtggagccagtacaccacatcactttcccagtttacccaagccccaccttctctaggcaccagttcaattgcccacccctccccccaacttctcagggactgtgggccatgtgctctctgcccactgaggggcactcagccctcaagcatgctcttctccactagtcacccctattgaccttatgtatgtgccaataatgggaaaaacccattgactcaccccctattgaccttttgtactgggcaaaacccaatggaaagtccctattgactcagtgtacttggctccaatgggactttcctgttgattggcgcgcccgggggatccagtttggttaattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctctagagtcgagaattcggccatgcgtcccctgcgcccccgcgccgcgctgctggcgctcctggcctcgctcctggccgcgcccccggtggccccggccgaggccccgcacctggtgcaggtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatgagcctgtgctgagccccagtgggatcctctagagtcgagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcataccggtcaagctagcgatatcaattaaccctcactaaagggagaccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatctggtaccatttaaatctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttttgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgca